(19) United States
(12) Patent Application Publication
(10) Pub. No.: US 2012/0148547 A1
(43) Pub. Date: Jun. 14, 2012

(54) COMBINATION OF VITAMIN E AND BETA-GLYCOSPHINGOLIPIDS IN COMPOSITIONS AND METHODS FOR PREVENTING AND TREATING HEPATIC DISORDERS

(75) Inventors: Yaron Ilan, Jerusalem (IL); Meir Mizrahi, Modi'in (IL); Ami Ben-Ya'acov, Jerusalem (IL)

(73) Assignee: Hadasit Medical Reasearch Services & Development Ltd., Jerusalem (IL)

(21) Appl. No.: 13/391,005

(22) PCT Filed: Aug. 31, 2010
(86) PCT No.: PCT/IL2010/000713

§ 371 Date: Feb. 17, 2012

Related U.S. Application Data
(60) Provisional application No. 61/238,702, filed on Sep. 1, 2009.

(51) Int. Cl. A61K 31/7032; A61K 35/12; A61P 9/10; A61P 29/00; A61P 1/16

(52) U.S. Cl. 424/93.7; 514/25

(57) ABSTRACT

The present invention relates to combined compositions comprising a combination of at least one natural or synthetic beta-glycolipid and at least one tocopherol (vitamin E). The invention further provides methods and kits using said combined compositions for treating and preventing hepatic disorders, specifically, liver insult cause by hepatotoxic drugs or caused by any one of infectious, metabolic, toxic, immune, or perfusion or blood flow related hepatic injury.

### FIELD OF THE INVENTION

The present invention relates to combined therapy of vitamins and glycolipids. More particularly, the invention provides compositions and methods for treating and preventing hepatic disorders, specifically, liver insult cause by hepatotoxic drugs or caused by any one of infectious, metabolic, toxic, immune, or perfusion or blood flow related hepatic injury.

### BACKGROUND OF THE INVENTION

All publications mentioned throughout this application are fully incorporated herein by reference, including all references cited therein.

Drug hepatotoxicity or drug induced liver injury (DILI) accounts for around 2 to 5 percent of patients requiring hospitalization for jaundice, and 10 percent of cases of hepatitis in all adults and more than 40 percent in patients elder than 50. The general occurrence is between one in 10,000 to 100,000 [Sgro, C. et al. Hepatology; 36:451-5 (2002)]. Drug hepatotoxicity is furthermore the most common reason of acute liver failure in the United States [Ostapowicz, G. et al. Ann. Intern. Med. 137:947 (2002)]. Hepatotoxicity can take place with different type of drugs due to a variety of mechanisms.

The liver is one of the main organs responsible for concentrating and metabolizing a major part of drugs and toxins that are introduced into the eukaryotic organism. These compounds are metabolized by a large number of soluble and membrane-bound enzymes, especially those associated with the hepatocyte endoplasmic reticulum. Every drug has its precise enzyme disposal pathway(s) of biotransformation involving one or more of these enzyme systems. It has been found that dissimilarity in drug metabolism might influence the development of drug toxicity in some individuals. Most drugs and toxins are excreted by the kidney and bile and, therefore, processes involved with extraction require solubility of the drugs. Nearly all of oral drugs absorbed from the gastrointestinal tract are lipophilic and water-insoluble. The lipophilic and water-soluble processes take place in the hepatic cells and thus more effortlessly excreted [Park, B. K. et al. Pharmacol. Ther. 68:385 (1995)]. Exogenous products are metabolized in the liver, mainly, via the mechanisms of phase I and phase II reactions.

During phase I metabolism, in order to facilitate water-solubility, polar groups are added to lipophilic molecules by variety of mechanisms including oxidation, reduction, or hydrolysis. This group of reactions is catalyzed by the cytochrome P450 super-family of mixed function oxidases (CYP). This family is found in the majority of cases on the cytoplasmic side of the membrane of the endoplasmic reticulum of the centrilobular (zone 3) hepatocytes [Nelson, D. R. et al. DNA Cell Biol. 12:1 (1993)]. These membrane-bound hemoproteins are composed of an apoprotein and a heme prosthetic group (oxidizing center) and operate in conjunction with NADPH.

Three families (CYP1, CYP2, and CYP3) are assumed to be the most important for hepatic metabolism of exogenous drugs and toxins [Ehrenpreis, E. D. and Ehrenpreis, S. Clin. Liver Dis. 2:457 (1998)]. The majority of drugs and toxins (such as cyclosporine, erythromycin, ketoconazole, lidocaine, phenobarbital, and phenyloin) are metabolized by the CYP3A subfamily [Wilkinson, G. R. J. Pharmacokinet. Biopharm. 24:475 (1996)].

Many factors can alter the activity of the CYP enzyme and therefore may potentially increase the toxicity of a compound (either by reducing its conversion to nontoxic metabolites or by increasing its conversion to toxic metabolites) or decrease its therapeutic effectiveness (e.g., by increasing the rate of metabolism of active drug) [Walgren, J. L. et al. Crit. Rev. Toxicol. 35:325 (2005)]. In various cases, alternate detoxification may grow to be the cause of hepatotoxicity. This may in part explain why some drugs (acetaminophen, for example) are not toxic in normal therapeutic doses but are toxic when increased amounts are ingested.

Following phase I metabolism, most compounds are still inadequate for excretion and require further metabolism. Phase II reactions result in the formation of readily extractable, nontoxic substances [Park, B. K. et al. Pharmacol. Ther. 68:385 (1995)] by conjugation of the drug metabolite to a large water-soluble polar group, such as glucuronic acid, sulfate, acetate, glycine, glutathione, or a methyl group. These processes occur mainly within the hepatocyte cytoplasm via the UDP-glucuronyl transferases, sulfotransferases, and glutathione S-transferases. These enzymes are rarely responsible for toxic metabolite formation, and their nontoxic products are usually ready for excretion [Nelson, S. D. Drug-Induced Liver Disease, Kaplowitz, N. DeLeve, L D. (Eds), Marcel Dekker, New York: p. 287 (2003)].

Several factors can alter the activity of either phase I or phase II reactions and influence drug metabolism. Induction of CYP enzymes has been observed with the ingestion of some foods, for example. CYP activity may be affected also by protein intake and the states of nourishment. High protein diets increased CYP activity, and CYP activity is reduced by low protein diets and severe malnutrition [Zhang, W. et al. Eur. J. Drug Metab. Pharmacokinet, 24:141 (1999)]. Chronic alcohol consumption increases the activity of CYP2E1 two-fold and depletes glutathione levels, resulting in lower defense of this compound against toxic metabolites [Prescott, L. F. Br. J. Clin. Pharmacol. 49:291 (2000)]. Among the drugs that have increased hepatotoxicity when associated with alcohol intake are acetaminophen, isoniazid, cocaine, methotrexate, and vitamin A [Lewis, J. H. Clin. Prac. Gastroenterol. Brandt, L. J. (Ed), Churchill Livingstone, Philadelphia: p. 855 (1998)]. States of severe malnutrition or chronic alcoholism may influence certain detoxifying cofactors such as glutathione.

The parallel use of more then one drug may be one of the most important factors affecting components of the CYP system and influencing drug metabolism. A drug may have the capacity to inhibit or enhance another drug's metabolism [Flockhart, D. A. and Oesterheld, J. R. Child. Adolesc. Psychiatr. Clin. N. Am. 9:43 (2000)]. Competitive inhibition of CYP can lead to clinically important drug interactions, for example the development of torsade de pointes (a specific variety of ventricular tachycardia) during the administration of terfenadine or cisapride to a patient taking a CYP3A4 inhibitor such as erythromycin or ketoconazole. Reduced phase II reactions are not common but have been described with the use of chlorpromazine and valproic acid.

Aging can also lead to a decrease in CYP activity [Hunt, C. M. Mech. Ageing Dev. 64:189 (1992)]. This phenomenon has been observed in elderly patients with the metabolism of acetaminophen, isoniazid, verapamil, nifedipine, lidocaine, and propranolol [Park, B. K. et al. Pharmacol. Ther. 68:385 (1995)]. Phase II enzymes do not appear to be altered by aging. However, albumin production may be reduced in the elderly, which may lead to increased availability of free drug for phase I or II metabolism.

Genetic polymorphisms in the CYP isoenzymes are seen in a large part of the population [Smith, G. et al. Xenobiotica, 28:1129 (1998)]. These genetic differences may add to either diminished metabolism, lack of metabolism, or excessive metabolism of a compound [Ueshima, Y. et al. Clin. Exp. Res. 20:25A (1996)]. This genetic variability may explain some of the individual hypersensitivity reactions to specific drugs. Genetic polymorphisms in the phase II enzymes lead to both decreased and increased activity. This is observed in glutathione s-transferases and the hepatotoxicity seen with certain chemical carcinogens [Seidegard, J. et al. Carcinogenesis 11:33 (1990)].

Both acute and chronic liver diseases have a variable effect on the Phase I metabolism of many drugs. Depending on the severity of liver dysfunction, CYP activity may be unaltered, or greatly reduced [Ehrenpreis, E. D. and Ehrenpreis, S. Clin. Liver Dis. 2:457 (1998)]. The type of liver disease does not appear to be important. Phase II enzyme activity does not appear to be distorted in most liver diseases, and enzyme activity may actually increase in severe liver disease [Debinski, H. S. et al. Gastroenterology 108:1464 (1995)].

The mechanisms of drug induced hepatotoxicity lead to hepatocyte necrosis, but there are some other mechanisms, such as damage the bile ducts or canaliculi (resulting in cholestasis), vascular endothelial cells (producing venoocclusive disease), or the stellate cells. There may also be mixed patterns of injury [Holt, M. P. and Ju, C. AAPS J. 8:E48 (2006)].

Toxic hepatocellular injury may be divided into two broad groups: direct chemical reactions (intrinsic hepatotoxins), and idiosyncratic reactions or immune-mediated hypersensitivity.

Intrinsic hepatotoxins reproducibly cause dose-dependent hepatocellular necrosis (“toxic” hepatitis). The latent period from the exposure to the drug and onset of the reaction is brief. Serum amino-transferases typically reach about eight to 500 folds of the normal levels, while serum alkaline phosphatase only reaches about one to two folds of the normal level [Lewis, J. H. Clin. Prac. Gastroenterol. Brandt, L. J. (Ed), Churchill Livingstone, Philadelphia: p. 855 (1998)]. The mortality is high in severe cases.

In a large part of intrinsic hepatotoxicity cases, the chemical composite itself or one of its active metabolites interact with multiple intracellular constituents to create a sequence of actions frequently resulting in cell damage and death. The mechanisms of injury and cell death are currently only partially understood. Production of free radicals, electrophilic radicals, or reactive oxygen species may be part of the cell injury and death. Alternatively, covalent binding of the toxic metabolite to structures within the cell may interfere with their function or their regulation.

Numerous cases of drug-related hepatotoxicity are associated with idiosyncratic reactions. The principal characteristic of this type of reaction is the apparent unpredictability of injury in humans. The reactions are species-specific and cannot be reproduced experimentally in laboratory animals. Idiosyncrasy may be either immunologic (hypersensitivity) or metabolic. The injurious reaction to a drug may be classified as a hypersensitivity reaction if it is accompanied by clinical and histologic evidence of classic hypersensitivity and is a result of using numerous medications such as phenyloin, amoxicillin-clavulanate, dihydralazine, sulfonamides, halothane, dapsone, diclofenac, carbamazepine, and sulindac) [Zimmerman, H. J. Schiffs Diseases of the Liver, Schiff, E. Sorrell, M. Maddrey, W. (Eds), Lippincott-Raven, Philadelphia, Pa.: p. 973 (1999)].

Genetic factors such as the modification of “self” due to covalent binding of the active metabolite with host tissues can influence drug-induced hypersensitivity and may lead to allergic reaction [Kenna, J. G. et al. J. Pharmacol. Exp. Ther. 245:1103 (1988)]. Genetic polymorphisms of the major histocompatibility molecules (HLA) can also influence the hypersensitivity reaction; for example, HLA-DR6 is seen in hepatitis due to chlorpromazine, and HLA-A11 is seen in hepatitis from tricyclic antidepressants [Larrey, D. et al. Gut, 33:368 (1992)].

The metabolic type of hepatic injury is most likely due to abnormal metabolism of the drug in risk patients. The metabolic hepatic injury reflects the tendency of a patient to produce toxic metabolites from a compound to a greater degree than other individuals. Drugs in this category include isoniazid, ketoconazole, diclofenac, disulfiram, valproate, troglitazone, and amiodarone.

The location of atypical metabolism of the drug is most likely the hepatocyte. Confined accumulation of toxic metabolites causes binding to cell proteins, and leads to cellular necrosis [Lewis, J. H. Clin. Prac. Gastroenterol. Brandt, L J (Ed), Churchill Livingstone, Philadelphia: p. 855 (1998)]. Immunologic injury may also play a role. Neoantigens may be formed by the reaction of the metabolite with the hepatocyte, leading to activation of the immune system [Spielberg, S. P. et al. N. Engl. J. Med. 305:722 (1981)].

Drug-induced liver injury (DILI) can be classified based upon the clinical appearance and laboratory features, the mechanism of toxicity, and/or the histological findings [Chang, C. Y. and Schiano, T. D. Aliment. Pharmacol. Ther. 25:1135 (2007)]. Acute presentations have large spectrum from asymptomatic mild biochemical abnormalities to an acute illness with jaundice that resembles viral hepatitis to acute liver failure [Chang, C. Y. and Schiano, T. D. Aliment. Pharmacol. Ther. 25:1135 (2007]. The presence of jaundice (serum bilirubin >3 times the upper limit of normal) in association with aminotransferase elevations is associated with a worse prognosis than isolated aminotransferase abnormalities (“Hy's law”) [Chitturi, S, and George, J. Semin. Liver Dis. 22:169 (2002]. In addition to these acute hepatitic presentations, some drugs are related to chronic histologic inflammatory changes and a clinical syndrome resembling autoimmune hepatitis, while others cause endothelial damage or thrombosis leading to vascular complications such as veno-occlusive disease or Budd-Chiari syndrome. Removal of the drug usually leads to “healing” of the injury. On the other hand, some types of toxicity can be associated with a progressive course, probably leading to fibrosis or cirrhosis, despite discontinuation of the drug.

DILI due to the use of acetaminophen has become the most common cause of acute liver failure in the United States [Ostapowicz, G. et al. Ann. Intern. Med. 137:947 (2002)]. The toxic dose of acetaminophen may vary among individuals according to baseline glutathione levels and other factors, it is unlikely that a single dose of less than 150 mg/kg in a child or 7.5 to 10 g for an adult results in hepatic toxicity. Almost all patients who consume doses in excess of 350 mg/kg develops severe liver toxicity (defined as peak aspartate aminotransferase (AST) or alanine aminotransferase (ALT) levels greater than 1000 IU/L) unless correctly treated [Prescott, L F. Drugs 25:290 (1983)].

With the ingestion of therapeutic doses, 90 percent of acetaminophen is metabolized in the liver to sulfate and glucuronide conjugates, which are then excreted in the urine [Forrest, J. A. et al. Clin. Pharmacokinet. 7:93 (1982)]. Around two percent is excreted in the urine unchanged. The remaining acetaminophen is metabolized via the hepatic cytochrome P450 (CYP2E1, CYP1A2, CYP3A4 subfamilies) mixed function oxidase pathway into a toxic, highly reactive, electrophilic intermediate, N-acetyl-p benzoquinoneimine (NAPQI) [Manyike, P. T. et al. Clin. Pharmacol. Ther. 67:275 (2000)].

Appropriate acetaminophen doses create a small amount of NAPQI which is quickly conjugated with hepatic glutathione, forming nontoxic cysteine and mercaptate compounds that are excreted in the urine [Mitchell, J. R. et al. J. Pharmacol. Exp. Ther. 187:211 (1973)]. On the other hand, with toxic doses of a acetaminophen the sulfation and glucuronidation pathways are saturated, and more acetaminophen is metabolized to NAPQI via the cytochrome P450 enzymes [Linden, C. H. and Rumack, B. H. Emerg. Med. Clin. North. Am. 2:103 (1984)]. When hepatic glutathione stores are used up by about 70 to 80 percent, NAPQI begins to react with hepatocytes, and injury ensues [Prescott, L F. Drugs 25:290 (1983)]. NAPQI arylates and binds covalently to the cysteine groups on hepatic macromolecules, forming NAPQI-protein adducts [James, L. P. et al. Drug. Metab. Dispos. 31:1499 (2003)]. This process is permanent and leads to oxidative injury and hepatocellular centrilobular necrosis. Although not entirely characterized, lipid peroxidation and mitochondrial injury likely play a role in the progression of hepatocellular injury [Larson, A. M. et al. Hepatology; 42:1364 (2005); Knight, T. R. et al. Toxicol. Sci. 76:229 (2003)]. Additionally, it appears that the release of cytokines and reactive nitrogen and oxygen species from damaged hepatocytes also play a role in the spread of hepatic injury. Cytokine liberate from hepatocytes may initiate a secondary inflammatory response from Kupffer cells and other inflammatory cells such as the innate immune system and neutrophils, extending the zone of hepatic injury [Michael, S. L. et al. Hepatology, 30:186 (1999)].

The present invention provides a novel combination of β-glycolipides, specifically, GC and Vitamin E. The invention further provides uses of this novel composition for treating and preventing hepatic disorders.

More specifically, the present invention firstly demonstrates reduction of liver injury due to acetaminophen ingestion, by promotion of LAP+ regulatory T lymphocytes and the glutathione system. The invention further shows that the use of combination of glucosylceramide (GC) and vitamin E serve as a tool for protecting liver from acetaminophen insult due to oxidative stress and innate immune system.

Thus, one object of the invention is to provide solutions to the unmet need of DILI by co-administration of a combination of GC and vitamin E, with the drug, as a preventive composition or alternatively, as a therapeutic composition after DILI is already developed.

The invention further provides the generation of “Safe drug” based on combining the glycosphingolipids (GC)+ vitamin E with the drug, specifically, acetaminophen.

These and other objects of the invention will become apparent as the description proceeds.

### SUMMARY OF THE INVENTION

According to a first aspect, the invention relates to a composition comprising a combination of at least one natural or synthetic beta-glycolipid and at least one tocopherol (vitamin E), tocotrienol or any derivatives thereof. The composition of the invention may optionally further comprises at least one additional therapeutic agent and at least one pharmaceutically acceptable carrier, diluent, excipient and/or additive. Specific combination of the invention comprises glucosylceramide (GC) and alpha-tocopherol, creating the GC-vitamin E combined composition.

According to one specific embodiment, the invention provides a combined composition further comprising at least one additional therapeutic agent selected from analgesic or antipyretic drug. Such drug may be for example, acetaminophen, and therefore the invention provides a composition of safe drug.

The invention further provides a pharmaceutical composition for treating, preventing, ameliorating, reducing or delaying the onset of acute or, chronic toxic effect of an analgesic or an antipyretic drug or any type of liver insult selected from infectious metabolic, toxic, immune, or perfusion or blood flow related hepatic injury, in a subject in need thereof. The pharmaceutical composition comprises as an active ingredient a therapeutically effective amount of the GC-vitamin E combination of the invention. According to one specific embodiment, the pharmaceutical composition of the invention is particularly applicable for treating, preventing, ameliorating, reducing or delaying the onset of drug induced liver injury (DILI), caused by acetaminophen.

Another aspect of the invention relates to a method of treating, preventing, ameliorating, reducing or delaying the onset of acute or chronic toxic effect of an analgesic or an antipyretic drug or any type of liver insult selected from infectious metabolic, toxic, immune, or perfusion or blood flow related hepatic injury, in a subject in need thereof. The method of the invention comprises the step of administering a therapeutically effective amount of a combination of at least one natural or synthetic beta-glycolipid and at least one tocopherol (vitamin E), tocotrienol or any derivatives thereof, before, simultaneously with, after or any combination thereof, administration of said drug to said subject.

According to one specific embodiment, the method of the invention is particularly applicable for treating, preventing, ameliorating, reducing or delaying the onset of drug induced liver injury (DILI), caused by acetaminophen.

A further aspect of the invention relate to the use of a therapeutically effective amount of a combination of at least one natural or synthetic beta-glycolipid, at least one tocopherol (vitamin E), tocotrienol or any derivatives thereof, an optionally at least one additional therapeutic agent, in the preparation of a medicament for treating, preventing, ameliorating, reducing or delaying the onset of acute or chronic toxic effect of an analgesic or an antipyretic drug or any type of liver insult selected from infectious metabolic, toxic, immune, or perfusion or blood flow related hepatic injury.

Another aspect of the invention relates to a kit for achieving a therapeutic effect in a subject suffering from acute or chronic toxic effect of an analgesic or an antipyretic drug or any type of liver insult selected from infectious metabolic, toxic, immune, or perfusion or blood flow related hepatic injury. According to one embodiment, the kit of the invention comprises:

(a) at least one natural or synthetic beta-glycolipid or a pharmaceutically acceptable derivative thereof and a pharmaceutically acceptable carrier or diluent, optionally in a first unit dosage form;
(b) at least one tocopherol (vitamin E), tocotrienol or any derivatives thereof, and a pharmaceutically acceptable carrier or diluent, optionally, in a second unit dosage form; and
(c) container means for containing said first and second dosage forms.

A further aspect of the invention relates to a method of inducing at least one T regulatory cell in a subject in need thereof, specifically, a subject suffering of acute or chronic effect of acetaminophen. The method of the invention comprises the step of administering to said subject a therapeutically effective amount of at least one of:

(a) a combination of at least one natural or synthetic beta-glycolipid and at least one tocopherol (vitamin E), tocotrienol or any derivatives thereof;
(b) an immune-cell treated with any one of (a) or with any composition comprising the same;
(c) an immune-cell obtained from a subject treated with any one of (a), (b) or any combination or mixtures thereof or any composition comprising the same; and
(d) a composition comprising any one of (a), (b), (c) or any combinations or mixtures thereof, said composition optionally further comprises at least one pharmaceutically acceptable carrier, diluent, excipient and/or additive.

Other aspects of the invention will become apparent by the hand of the following figures.

### BRIEF DESCRIPTION OF THE FIGURES

FIG. 1: GC reduces ALT levels of animals suffering of acetaminophen-induced liver injury

ALT serum levels were measured 12 hr after induction of liver injury by acetaminophen. Animals were treated with GC two hours prior to liver injury induction (B), and after induction (C). Control group (A) received no GC.

Abbreviations: ALT (alanine aminotransferase).

FIG. 2: GC reduces ALT levels of animals suffering acetaminophen-induced liver injury

ALT serum levels were measured 24 hr after induction of liver injury by acetaminophen. Animals were treated with GC two hours prior to liver injury induction (B), and after induction (C). Control group (A) received no GC.

Abbreviations: ALT (alanine aminotransferase).

FIG. 3: GC reduces AST levels of animals suffering of acetaminophen-induced liver injury

AST serum levels were measured 24 hr after induction of liver injury by acetaminophen. Animals were treated with GC two hours prior to liver injury induction (B), and after induction (C). Control group (A) received no GC.

Abbreviations: AST (aspartate aminotransferase).

FIG. 4: Combination of GC and different vitamins reduces ALT levels of animals suffering of acetaminophen-induced liver injury

ALT serum levels were measured 24 hr after induction of liver injury by acetaminophen. Animals were treated with vitamins C, E, C+E with or without GC. Control group received no GC or vitamins.

Abbreviations: N. GC (no glucosylceramide); GC (glucosylceramide); Cont. (control); Vit. (vitamin); ALT (alanine aminotransferase).

FIG. 5: Combination of GC and different vitamins reduces AST levels of animals suffering of acetaminophen-induced liver injury

AST serum levels were measured 24 hr after induction of liver injury by acetaminophen. Animals were treated with vitamins C, E, C+E with or without GC. Control group received no GC or vitamins.

Abbreviations: N. GC (no glucosylceramide); GC (glucosylceramide); Cont. (control); Vit. (vitamin); AST (aspartate aminotransferase).

FIG. 6: Synergistic combination of GC and vitamin E reduces ALT levels of animals suffering of acetaminophen-induced liver injury

ALT serum levels were measured 24 hr after induction of liver injury by acetaminophen. Animals were treated with GC, vitamin E, and a combination of vitamin E with GC. Control group received no GC or vitamin E.

Abbreviations: GC (glucosylceramide); Cont. (control); Vit. (vitamin); ALT ser. lev. (alanine aminotransferase serum level).

FIG. 7: Combination of GC and different vitamins elevates GSH serum levels of animals suffering of acetaminophen-induced liver injury

GSH serum levels were measured 24 hr after induction of liver injury by acetaminophen. Animals were treated with vitamins C, E, C+E with or without GC. Control group received no GC or vitamins.

Abbreviations: N. GC (no glucosylceramide); GC (glucosylceramide); Cont. (control); Vit. (vitamin); GSH (gluthathione-SH).

FIG. 8: Combination of GC and different vitamins reduce TNF-α serum levels in animals suffering of acetaminophen-induced liver injury

GSH serum levels were measured 24 hr after induction of liver injury by acetaminophen. Animals were treated with vitamins C, E, C+E with or without GC. Control group received no GC or vitamins.

Abbreviations: N. GC (no glucosylceramide); GC (glucosylceramide); Cont. (control); Vit. (vitamin); TNF-α (tumor necrosis factor α).

### DETAILED DESCRIPTION OF THE INVENTION

According to a first aspect, the invention relates to a composition comprising a combination of at least one natural or synthetic beta-glycolipid and at least one tocopherol (vitamin E), tocotrienol or any derivatives thereof. The composition of the invention may optionally further comprise at least one additional therapeutic agent and at least one pharmaceutically acceptable carrier, diluent, excipient and/or additive.

According to one embodiment, the beta-glycolipid comprised within the combined composition of the invention may be selected from the group consisting of a glucosylceramide, a monosaccharide ceramide, a galatosylceremide, a lactosyl-ceramide, a gal-gal-glucosyl-ceramide, GM2 ganglioside, GM3 ganglioside, globoside or any synthetic or natural β-glycolipid or any derivative or combination thereof.

In yet another embodiment, the tocopherol (vitamin E), tocotrienol or any derivatives thereof comprised within the combined composition of the invention may be selected from the group consisting of: alpha-tocopherol, beta-tocopherol, gamma-tocopherol, delta-tocopherol, alpha-tocotrienol, beta-tocotrienol, gamma-tocotrienol, delta-tocotrienol and any combination thereof.

Vitamin E is the general name for a class of eight compounds: four isomers of tocopherol and four isomers of tocotrienol. Structurally, tocopherols and tocotrienols share some resemblance consisting of common chromanol head and side chain at the C-2 position. Tocopherols and tocotrienols are sometimes collectively called tocols. Vitamin E is now considered a generic name describing bioactivities of both tocopherols and tocotrienols derivatives. Vitamin E is a fat-soluble vitamin necessary in the diet of many species for normal reproduction, normal development of muscles, resistance of erythrocytes to hemolysis and various biochemical functions. The most broadly acknowledged function of Vitamin E, whereby it is an antioxidant. The Vitamin E content in crude palm oil ranges between 600-1000 parts per million (ppm) and is a mixture of tocopherols (18-22%) and tocotrienols (78-82%).

According to one specific embodiment, the invention provides a composition comprising a combination of glucosylceramide (GC) as a beta-glycolipid and alpha-tocopherol as tocopherol (vitamin E), tocotrienol or any derivatives thereof, thus creating the GC-vitamin E combined composition.

According to one embodiment, the combined composition of the invention may comprise at least one beta-glycolipid combined with at least one tocopherol (vitamin E), tocotrienol or any derivatives thereof, at any quantitative ratio of between about 1:1 to 1000:1. It should be appreciated that any quantitative ratio of the combined compounds may be used. As a non-limiting example, a quantitative ratio used between any of the compounds may be: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1500, 1:750, 1:1000. It should be further noted that where the combination of the invention comprises more than two compounds, the quantitative ratio used may be for example, 1:1:1, 1:2:3, 1:10:100, 1:10:100:1000 etc.

According to another particular and specific embodiment, the combination used by the compositions, methods and kits of the invention described herein later, may comprise between about 0.0001 to 100 mg per kg, specifically, 1.5 mg per kg of body weight β-glucosylceramide and between about 0.0001 to 100 mg per kg, specifically, 2.5 gr per kg of body weight vitamin E. More specifically, the combination used by the compositions, methods and kits of the invention described herein later, may comprise between about 0.0001 to 100 mg per kg, more specifically between about 0.001 to 90 mg per kg, more specifically between about 0.01 to 80 mg per kg, more specifically between about 0.1 to 80 mg per kg, more specifically between about 0.2 to 75 mg per kg, more specifically between about 0.3 to 70 mg per kg, more specifically between about 0.4 to 65 mg per kg, more specifically between about 0.5 to 60 mg per kg, most specifically, 0.5 mg per kg of body weight β-glucosylceramide, more specifically between about 0.6 to 55 mg per kg, more specifically between about 0.7 to 50 mg per kg, more specifically between about 0.8 to 45 mg per kg, more specifically between about 0.9 to 40 mg per kg, more specifically between about 1.0 to 35 mg per kg, more specifically between about 1.0 to 30 mg per kg, more specifically between about 1.0 to 25 mg per kg, more specifically between about 1.0 to 20 mg per kg, more specifically between about 1.0 to 15 mg per kg, more specifically between about 1.0 to 10 mg per kg, more specifically between about 1.0 to 9 mg per kg, more specifically between about 1.0 to 8 mg per kg, more specifically between about 1.0 to 7 mg per kg, more specifically between about 1.0 to 6 mg per kg, more specifically between about 1.0 to 5 mg per kg, more specifically between about 1.0 to 4 mg per kg, more specifically between about 1.0 to 3 mg per kg, most specifically, 1.5 mg per kg of body weight β-glucosylceramide, and between about 0.0001 to 100 mg per kg, more specifically between about 0.001 to 90 mg per kg, more specifically between about 0.01 to 80 mg per kg, more specifically between about 0.1 to 80 mg per kg, more specifically between about 0.2 to 75 mg per kg, more specifically between about 0.3 to 70 mg per kg, more specifically between about 0.4 to 65 mg per kg, more specifically between about 0.5 to 60 mg per kg, more specifically between about 0.6 to 55 mg per kg, more specifically between about 0.7 to 50 mg per kg, more specifically between about 0.8 to 45 mg per kg, more specifically between about 0.9 to 40 mg per kg, more specifically between about 1.0 to 35 mg per kg, more specifically between about 1.0 to 30 mg per kg, more specifically between about 1.0 to 25 mg per kg, more specifically between about 1.0 to 20 mg per kg, more specifically between about 1.0 to 15 mg per kg, more specifically between about 1.0 to 10 mg per kg, more specifically between about 1.0 to 9 mg per kg, more specifically between about 1.0 to 8 mg per kg, more specifically between about 1.0 to 7 mg per kg, more specifically between about 1.0 to 6 mg per kg, more specifically between about 1.0 to 5 mg per kg, more specifically between about 1.0 to 4 mg per kg, more specifically between about 1.0 to 3 mg per kg, most specifically, 2.5 gr per kg of body weight vitamin E.

As also shown by the following Examples, according to one specific embodiment, the toxicity ameliorating combined composition of the invention leads to a decrease or reduction in AST and ALT serum levels that are markers for liver damage. Such decrease or reduction according to the invention may be a reduction of about 5% to 99%, specifically, a reduction of about 5%, 10%, 15%, 20%, 25%, 30%, 35%, 40%, 45%, 50%, 55%, 60%, 65%, 70%, 75%, 80%, 85%, 90%, 95% or 99% as compared to untreated control.

According to one specific embodiment, the invention provides a combined composition further comprising in addition to the GC and vitamin E, also at least one additional therapeutic agent selected from analgesic or antipyretic drug. Such analgesic or antipyretic drug may be according to certain embodiments, an inducer or inhibitor of Cytochrom P450 selected from the group consisting of: Acetaminophen, Phenobarbital, Phenyloin, Carbamazepine, Primidone, Ethanol, Glucocorticoids, Rifampin, Griseofulvin, Quinine, Omeprazole, Amiodarone, Cimetidine, Erythromycin, Grape fruit, Isoniazid, Ketoconazole, Metronidazole, Sulfonamides, Chlorpromazine, phenylbutazone, halogenated anesthetic agents, sulindac, Dapsone, INH, halothane, amoxicillin-clavulanic acid, phenobarbital, Para-amino salicylate, Clofibrate, Procainamide, Gold salts, propylthiouracil, chloramphenicol, nitrofurantoin, methoxyflurane, penicillamine, paraquat, Tetracycline, Contraceptive and anabolic steroids, rifampin, Aspirin and Sodium valproate. According to one specific embodiment, the invention relates to a combined composition comprising GC, vitamin E and acetaminophen, thereby providing a safe preparation of acetaminophen, having reduced potential for hepatic toxicity.

Therefore, the invention further provides a pharmaceutical composition for treating, preventing, ameliorating, reducing or delaying the onset of acute or chronic toxic effect of an analgesic or an antipyretic drug in a subject in need thereof. Moreover, the pharmaceutical composition of the invention may be used for treating and preventing any type of liver insult selected from infectious metabolic, toxic, immune, or perfusion or blood flow related hepatic injury. The pharmaceutical composition of the invention comprises as an active ingredient a therapeutically effective amount of a combination of at least one natural or synthetic beta-glycolipid and at least one tocopherol (vitamin E), tocotrienol or any derivatives thereof, and optionally at least one additional therapeutic agent, with a pharmaceutically acceptable carrier.

According to one embodiment, the pharmaceutical composition of the invention is intended for treating, preventing, ameliorating, reducing or delaying the onset of acute or chronic toxic effect of analgesic or antipyretic drug. Such drug may be according to certain embodiments, an inducer or inhibitor of Cytochrom P-450 selected from the group consisting of: Acetaminophen, Phenobarbital, Phenyloin, Carbamazepine, Primidone, Ethanol, Glucocorticoids, Rifampin, Griseofulvin, Quinine, Omeprazole, Amiodarone, Cimetidine, Erythromycin, Grape fruit, Isoniazid, Ketoconazole, Metronidazole, Sulfonamides, Chlorpromazine, phenylbutazone, halogenated anesthetic agents, sulindac, Dapsone, INH, halothane, amoxicillin-clavulanic acid, phenobarbital, Para-amino salicylate, Clofibrate, Procainamide, Gold salts, propylthiouracil, chloramphenicol, nitrofurantoin, methoxyflurane, penicillamine, paraquat, Tetracycline, Contraceptive and anabolic steroids, rifampin, Aspirin and Sodium valproate.

According to one specific embodiment, the pharmaceutical composition, the combined composition and kit of the invention is intended for treating, preventing, ameliorating, reducing or delaying the onset of acute or chronic toxic effect of the analgesic drug N-(4-hydroxyphenyl)ethanamide, known as acetaminophen (paracetamol).

N-(4-hydroxyphenyl)ethanamide Paracetamol or acetaminophen is a widely used over-the-counter analgesic (pain reliever) and antipyretic (fever reducer). It is commonly used non-steroidal analgesic agent for the relief of fever, headaches, and other minor aches and pains, and is a major ingredient in numerous cold and flu remedies.

While acetaminophen has fewer gastro-intestinal side effects than aspirin, another commonly used non-steroidal analgesic agent, acute and chronic acetaminophen toxicity can result in gastro-intestinal symptoms, severe liver damage, and even death. The precise intermediates in the acetaminophen toxic metabolite pathway are not yet known. As indicated herein before, it had been thought that when acetaminophen was ingested, the cytochrome P-450 dependent enzyme system of the liver produced a potentially toxic metabolite of acetaminophen which was the cause of acetaminophen toxicity.

It was further believed that when safe amounts of acetaminophen had been ingested, this toxic metabolite was cleared by hepatic glutathione stores. However in the case of acute or chronic overdose, excessive levels of the toxic metabolite were thought to delete the glutathione stores in the liver, resulting in hepatic necrosis. Later studies have proposed that acetaminophen induced hepatic necrosis may be due to cellular oxidative stress, resulting both in lipid peroxidation, protein and non-protein thiol oxidation, and changes in the intracellular calcium homeostasis. Symptoms of acute acetaminophen toxicity are typically mild or non-existent until at least 48 hours post-ingestion.

Thus, in yet another embodiment the acute or chronic toxic effect of acetaminophen treated by the combined composition of the invention may be any one of drug induced liver injury (DILI), drug-induced acute steatosis, cytotoxic hepatocellular injury, acute liver failure (ALF), reperfusion injury, ischemic liver disease and acute cholestatic injury.

According to one specific embodiment, the pharmaceutical combined composition of the invention is particularly applicable for treating, preventing, ameliorating, reducing or delaying the onset of drug induced liver injury (DILI), caused by acetaminophen.

It should be appreciated that the different Cytochrome P-450 inducing or inhibiting drugs may lead to different hepatic injuries, and therefore, may be prevented or treated by the combined compositions of the invention. For example, chlorpromazine, phenylbutazone, halogenated anesthetic agents and sulindac may cause fever, rash and eosinophilia. Dapsone may lead to sulfone syndrome (i.e., fever, rash, anemia, and jaundice), INH (Isoniazid (Laniazid, Nydrazid), also known as isonicotinylhydrazine (INH) and halothane may cause acute viral hepatitis, Chlorpromazine, erythromycin, amoxicillin- and clavulanic acid may lead to obstructive jaundice. Phenyloin, carbamazepine, Phenobarbital and primidone may cause anticonvulsant hypersensitivity syndrome (i.e., triad of fever, rash, and liver injury), Para-amino salicylate, phenyloin, sulfonamides, may lead to serum sickness syndrome, Clofibrate may lead to Muscular syndrome (i.e., myalgia, stiffness, weakness, elevated creatine kinase level), Procainamide may cause Antinuclear antibodies (ANAs), Gold salts, propylthiouracil, chlorpromazine and chloramphenicol may cause marrow injury. Drugs such as Amiodarone and nitrofurantoin may be lead to associated pulmonary injury and Gold salts, methoxyflurane, penicillamine, paraquat may also lead to Associated renal injury. Tetracycline may cause Fatty liver of pregnancy, Contraceptive and anabolic steroids and rifampin may cause bland jaundice, Aspirin may cause Reye syndrome, and Sodium valproate may lead to Reye like syndrome.

Still further, other acute hepatocellular injuries caused by drugs may be treated or prevented by the combined composition of the invention. For example, acute viral hepatitis-like picture may be caused by INH, halothane, diclofenac and troglitazone. Mononucleosis like picture may be a result of using phenyloin, sulfonamides or dapsone. Chronic hepatocellular injury may be a result of Pemoline or methyldopa. Massive necrosis may be a result of using acetaminophen, halothane or diclofenac.

Steatosis may also be a result of using different drugs, for example, Macro vesicular steatosis may be caused by Alcohol, methotrexate, corticosteroids, minocycline, nifedipine and TPN, Microvesicular steatosis may be caused by alcohol, valproic acid, tetracycline and piroxicam. Steatohepatitis may be a result of Amiodarone, nifedipine, synthetic estrogens and didanosine. Pseudoalcoholic injury may be caused by Amiodarone, Acute cholestasis maybe a result of using Amoxicillin-clavulanic acid, erythromycin and sulindac. Chronic cholestasis may be caused by Chlorpromazine, sulfamethoxazole-trimethoprim, tetracycline or ibuprofen. Granulomatous hepatitis may be a result of using Carbamazepine, allopurinol and hydralazine. Vascular injury may be caused by steroids, Neoplasia may be a result of using Contraceptives or anabolic steroids. Adenoma may be caused by steroids, Angiosarcoma may be a result of Vinyl chloride. Hepatocellular carcinoma may be caused by Anabolic steroids, aflatoxin, arsenic or vinyl chloride.

More particularly, a drug such as Amoxicillin may cause Hepatic dysfunction including jaundice, hepatic cholestasis, and acute cytolytic hepatitis.

In certain embodiments, the combined compositions of the invention may be applicable for preventing hepatic damage caused by a drug such as amiodarone. This drug may lead to abnormal liver function, as indicated by test results in 15-50% of patients. The spectrum of liver injury is wide, ranging from isolated asymptomatic transaminase elevations to a fulminant disorder. Hepatotoxicity usually develops more than one year after starting therapy, but it can occur in one month. It is usually predictable, dose dependent, and has a direct hepatotoxic effect. Some patients with elevated aminotransferase levels have detectable hepatomegaly, and clinically important liver disease develops in less than 5% of patients. In rare cases, amiodarone toxicity manifests as alcoholic liver disease. Hepatic granulomas are rare. Importantly, amiodarone has a very long half-life and therefore may be present in the liver for several months after withdrawal of therapy. Since amiodarone is iodinated, it results in increased density on CT scans, which does not correlate with hepatic injury.

Still further, the combined composition of the invention may also be applicable in cases of using drug such as Chlorpromazine. This drug may lead to liver injury that resembles that of infectious hepatitis with laboratory features of obstructive jaundice rather than those of parenchymal damage. The overall incidence of jaundice is low regardless of dose or indication of the drug. Most cases occur two to four weeks after therapy. Any surgical intervention should be withheld until extrahepatic obstruction is confirmed. It is usually promptly reversible upon withdrawal of the medication; however chronic jaundice has been reported. Chlorpromazine should be administered with caution to persons with liver disease.

A further embodiment of the invention provides the use of the combined compositions of the invention for preventing or treating liver damage caused by ciprofloxacin. Cholestatic jaundice has been reported with repeated use of quinolones. Approximately 1.9% of patients taking ciprofloxacin show elevated SGPT (Serum glutamic pyruvic transaminase, an enzyme that is normally present in liver and heart cells) levels, 1.7% showed elevated SGOT (Serum glutamic oxaloacetic transaminase) levels, 0.8% have increased alkaline phosphatase levels, and 0.3% showed elevated bilirubin levels.

Also a drug such as Diclofenac exhibits a variety of potential liver injuries that may be treated or prevented by the combined composition of the invention. Elderly females are more susceptible to diclofenac-induced liver injury. Elevations of one or more liver test results may occur. These laboratory abnormalities may progress, may remain unchanged, or may be transient with continued therapy. Borderline or greater elevations of transaminase levels occur in approximately 15% of patients treated with diclofenac. Of the hepatic enzymes, ALT is recommended for monitoring liver injury. Meaningful (>3 times the upper limit of the reference range) elevations of ALT or AST occur in approximately 2% of patients during the first 2 months of treatment. In patients receiving long-term therapy, transaminase levels should be measured periodically within 4-8 weeks of initiating treatment. In addition to the elevation of ALT and AST levels, cases of liver necrosis, jaundice, and fulminant hepatitis with and without jaundice have reported. As shown by the following Examples, the combined composition of the invention ameliorates liver damage, as manifested by reduction in AST and ALT levels. Therefore, such combined composition may be also used for diclofenac induced liver damage.

It should be further appreciated that the combined composition of the invention may also be used for preventing or treating liver damage caused by using Erythromycin. This drug may cause hepatic dysfunction, including increased liver enzyme levels and hepatocellular and/or cholestatic hepatitis with or without jaundice. A cholestatic reaction is the most common adverse effect and usually begins within 2-3 weeks of therapy.

Fluconazole is another example for a drug causing liver damage that may be prevented or treated by the combined composition of the invention. The spectrum of hepatic reactions ranges from mild transient elevations in transaminase levels to hepatitis, cholestasis, and fulminant hepatic failure. In fluconazole-associated hepatotoxicity, hepatotoxicity is not obviously related to the total daily dose, duration of therapy, or sex or age of the patient. Fatal reactions occur in patients with serious underlying medical illness.

Severe and fatal hepatitis has been reported with INH therapy. The risk of developing hepatitis is age related, with an incidence of 8 cases per 1000 persons older than 65 years. In addition, the risk of hepatitis is increased with daily consumption of alcohol. Mild hepatic dysfunction evidenced by a transient elevation of serum transaminase levels occurs in 10-20% of patients taking INH. This abnormality usually appears in the first 3 months of treatment, but it may occur anytime during therapy. In most instances, enzyme levels return to the reference range, with no need to discontinue the medication. Occasionally, progressive liver damage can occur, and therefore, the combined composition of the invention may be used also in preventing and treating NIH induced liver damage.

Methyldopa is a further example for a drug causing liver damage that may be prevented by the combined composition of the invention. Methyldopa is an antihypertensive that is contraindicated in patients with active liver disease. Periodic determination of hepatic function should be performed during the first 6-12 weeks of therapy. Occasionally, fever may occur within 3 weeks of methyldopa therapy, which may be associated with abnormalities in liver function test results or eosinophilia, necessitating discontinuation. In some patients, findings are consistent with those of cholestasis and hepatocellular injury. Rarely, fatal hepatic necrosis has been reported after use of methyldopa, which may represent a hypersensitivity reaction.

Oral contraceptives can lead to intrahepatic cholestasis with pruritus and jaundice in a small number of patients, and therefore may be treated by the combined composition of the invention. More specifically, patients with recurrent idiopathic jaundice of pregnancy, severe pruritus of pregnancy, or a family history of these disorders are more susceptible to hepatic injury. Oral contraceptives are contraindicated in patients with a history of recurrent jaundice of pregnancy. Benign neoplasm, rarely malignant neoplasm of the liver and hepatic vein occlusion have also been associated with oral contraceptive therapy.

Statins are among the most widely prescribed medications in the western world. The use of statins/HMG-CoA reductase inhibitors is associated with biochemical abnormalities of liver function, and thus may be also prevented or treated by the combined composition of the invention. Moderate elevations of serum transaminase levels (<3 times the upper limit of the reference range) have been reported following initiation of therapy and are often transient. Elevations are not accompanied by any symptoms and do not require interruption of treatment. Persistent increases in serum transaminase levels (>3 times the upper limit of the reference range) occur in approximately 1% of patients, and these patients should be monitored until liver function returns to normal after drug withdrawal. Active liver disease or unexplained transaminase elevations are contraindications to use of these drugs. Patients with a recent history of liver disease or persons who regularly consume alcohol in large quantities, should use statins in a regulated manner.

In certain embodiments, the combined compositions and kits of the invention may also be applicable for preventing and treating liver injury caused by Rifampin. Rifampin is usually administered with INH. On its own, rifampin may cause mild hepatitis, but this is usually in the context of a general hypersensitivity reaction. Fatalities associated with jaundice have occurred in patients with liver disease and in patients taking rifampin with other hepatotoxic agents. Careful monitoring of liver function (especially SGPT/SGOT) should be performed prior to therapy and then every 2-4 weeks during therapy. In some cases, hyper-bilirubinemia resulting from competition between rifampin and bilirubin for excretory pathways of the liver can occur in the early days of treatment. Isolated cholestasis also may occur.

In yet a further embodiment, the combined composition of the invention may be applicable for preventing or treating liver damage caused by Valproic acid and divalproex sodium. More specifically, microvesicular steatosis is observed with alcohol, aspirin, valproic acid, amiodarone, piroxicam, stavudine, didanosine, nevirapine, and high doses of tetracycline. Prolonged therapy with methotrexate, INH, ticrynafen, perhexyline, enalapril, and valproic acid may lead to cirrhosis. Valproic acid typically causes microsteatosis. This drug should not be administered to patients with hepatic disease and may be used with caution in patients with a prior history of hepatic disease. Those at particular risk include children younger than 2 years, those with congenital metabolic disorders or organic brain disease, and those with seizure disorders treated with multiple anticonvulsants.

Hepatic failures resulting in fatalities have occurred in patients receiving valproic acid. These incidents usually occur during the first six months of treatment and are preceded by nonspecific symptoms such as malaise, weakness, lethargy, facial edema, anorexia, vomiting, and even loss of seizure control.

It should be further appreciated that the combined composition of the invention may also be used for preventing or treating liver damage caused by using herbs. The increasing use of alternative medicines has led to many reports of toxicity. The spectrum of liver disease is wide with these medicines, for example: Senecio/crotalaria (Bush teas) can cause venoocclusive disease. Germander in teas is used for its anticholinergic and antiseptic properties. Jaundice with high transaminase levels may occur after two months of use, but it disappears after stopping the drug. Chaparral is used for a variety of conditions, including weight loss, cancer, and skin conditions. It may cause jaundice and fulminant hepatic failure. Chinese herbs have also been associated with hepatotoxicity.

According to certain embodiments, the combined composition of the invention may also be applicable in treating liver damage caused by recreational drugs. More specifically, Ecstasy is an amphetamine used as a stimulant and may cause hepatitis and cirrhosis. Cocaine abuse has been associated with acute elevation of hepatic enzymes. Liver histology shows necrosis and microvascular changes.

Still further, in certain embodiments the beta-glycolipid comprised within the pharmaceutical composition of the invention may be selected from the group consisting of a glucosylceramide, a monosaccharide ceramide, a galatosylceremide, a lactosyl-ceramide, a gal-gal-glucosyl-ceramide, GM2 ganglioside, GM3 ganglioside, globoside or any synthetic or natural β-glycolipid or any derivative or combination thereof, specifically, glucosylceramide (GC).

In yet another embodiment, the tocopherol (vitamin E), tocotrienol or any derivatives thereof used for the combined composition of the invention may be selected from the group consisting of: alpha-tocopherol, beta-tocopherol, gamma-tocopherol, delta-tocopherol, alpha-tocotrienol, beta-tocotrienol, gamma-tocotrienol, delta-tocotrienol and any combination thereof, specifically, alpha-tocopherol.

Thus, according to one specific embodiment, the invention provides a pharmaceutical composition comprising a combination of GC and alpha-tocopherol.

It should be further noted that the invention further provides a combined composition comprising GC, vitamin E and also the certain drug, for example, acetaminophen, that may be administered together as a safe composition preventing and reducing the hepatic damage that may be caused by acetaminophen.

Another aspect of the invention relates to a method of treating, preventing, ameliorating, reducing or delaying the onset of acute or chronic toxic effect of an analgesic or an antipyretic drug or any type of liver insult selected from infectious, metabolic, toxic, immune, perfusion or blood flow related hepatic injury in a subject in need thereof. The method of the invention comprises the step of administering a therapeutically effective amount of a combination of at least one natural or synthetic beta-glycolipid, specifically, GC and at least one tocopherol (vitamin E), tocotrienol or any derivatives thereof, before, simultaneously with, after or any combination thereof, administration of said drug to said subject.

It should be noted that the method of the invention is applicable to any liver damage associated or linked to acute or chronic toxic effect of an analgesic or an antipyretic drug. It should be further appreciated that the methods, kits and combined compositions of the invention may be applicable for treating analgesic or antipyretic drug-related conditions. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology.

According to one embodiment, the method of the invention is particularly applicable for treating, preventing, ameliorating, reducing or delaying the onset of acute or chronic toxic effect of analgesic or antipyretic drug, that may be an inducer or inhibitor of Cytochrom P-450 selected from the group consisting of: Acetaminophen, Phenobarbital, Phenyloin, Carbamazepine, Primidone, Ethanol, Glucocorticoids, Rifampin, Griseofulvin, Quinine, Omeprazole, Amiodarone, Cimetidine, Erythromycin, Grape fruit, Isoniazid, Ketoconazole, Metronidazole, Sulfonamides, Chlorpromazine, phenylbutazone, halogenated anesthetic agents, sulindac, Dapsone, INH, halothane, amoxicillin-clavulanic acid, phenobarbital, Para-amino salicylate, Clofibrate, Procainamide, Gold salts, propylthiouracil, chloramphenicol, nitrofurantoin, methoxyflurane, penicillamine, paraquat, Tetracycline, Contraceptive and anabolic steroids, rifampin, Aspirin and Sodium valproate.

According to one specific embodiment, the method of the invention is particularly applicable for treating, preventing, ameliorating, reducing or delaying the onset of acute or chronic toxic effect of analgesic drug such as acetaminophen (paracetamol).

According to another embodiment, acute or chronic toxic effect of acetaminophen treated by the method of the invention may be drug induced liver injury (DILI), drug-induced acute steatosis, cytotoxic hepatocellular injury, acute liver failure (ALF), reperfusion injury, ischemic liver disease and acute cholestatic injury.

According to one specific embodiment, the method of the invention is particularly applicable for treating, preventing, ameliorating, reducing or delaying the onset of drug induced liver injury (DILI), caused by acetaminophen.

DILI is usually considered subclinical or insignificant if the serum alanine aminotransferase (ALT) is <3 times the upper limit of normal [Watkins, P. B. and Seeff, L. B. Hepatology, 43:618 (2006)]. Subclinical liver disease has been described with the use of certain antibiotics, antidepressants, lipid-lowering drugs (such as simvastatin), sulfonamides, salicylates, sulfonylureas, and quinidine, usually in fewer than 5 to 10 percent of individuals [Tolman, K. G. Am. J. Cardiol. 89:1374 (2002); JAMA, 288:2998 (2002) (Dear inventor, pls. check this reference); Downs, J. R. et al. JAMA, 279:1615 (1998)]. A higher percentage of asymptomatic ALT elevations can be seen with other medications, including isoniazid (up to 20 percent) [Lewis, J. H. Clin. Prac. Gastroenterol. Brandt, L J (Ed), Churchill Livingstone, Philadelphia: p. 855 (1998); Monteith, D. K. et al. Drug. Chem. Toxicol. 19:71 (1996); Maddrey, W. C. Semin. Liver Dis. 1:129 (1981)]. Most subclinical ALT elevations are benign and resolve once the offending agent has been discontinued.

Acute DILI is the most common form of liver damage caused by drugs. The patterns of acute injury may present as hepatocellular (cytotoxic) damage, cholestasis, a mixed pattern of cytotoxic and cholestatic injury, or, a lesser amount of steatosis [Batt, A. M. and Ferrari, L. Clin. Chem. 41:1882 (1995)]. Discontinuation of the offending cause frequently results in complete recovery, even though the prognosis is generally poorer in patients with hepatocellular injury presenting with jaundice [Andrade, R. J. et al. Gastroenterology, 129:512 (2005)]. The most frequent drugs implicated in acute DILI in the United States are acetaminophen followed by antibiotics [Ostapowicz, G. et al. Ann. Intern. Med. 137:947 (2002); Galan, M. V. et al. J. Clin. Gastroenterol. 39:64 (2005)].

Drug-induced acute hepatocellular injury is analogous to that caused during viral hepatitis and includes hepatocellular necrosis or apoptosis, steatosis, and cellular degeneration. A typical finding on laboratory testing is an elevation in serum aminotransferases. Therefore, in certain embodiments the combined compositions, methods and kits of the invention may be applicable in treating any of these conditions. Hepatocyte that has become sensitized to the immune system dies by apoptosis via death receptors at the cell surface [Abboud, G. and Kaplowitz, N. Drug Saf. 30:277 (2007)]. Oxidative stress result in apoptosis at the intracellular level in moderate degree, at the same time a severe oxidative stress leads to necrotic cell lysis (necrosis).

The necrosis of the hepatic cells can be zonal or nonzonal, depending upon the offending cause. Zonal necrosis is characteristic of medications with predictable, dose-dependent, intrinsic toxicity, such as acetaminophen (zone 3), yellow phosphorus (mid-zonal), or iron sulfate (zone 1). There may be modest or no inflammatory response, yet, injured cells may accumulate fat (triglycerides). Non-zonal necrosis appears in a viral hepatitis-like model. It is more often seen with medications that produce unpredictable idiosyncratic injury (e.g., phenyloin, methyldopa, isoniazid, and diclofenac). Cytotoxic hepatocellular injury is related with a mortality rate of up to 10 percent overall and reach to 80 percent or higher if acute liver failure (ALF) develops [Ostapowicz, G. et al. Ann. Intern. Med. 137:947 (2002); Zimmerman, H. J. Schiffs Diseases of the Liver, Schiff, E. Sorrell, M. Maddrey, W. (Eds), Lippincott-Raven, Philadelphia, Pa.: p. 973 (1999); Speeg, K. V. and Bay, M. K. Gastroenterol. Clin. North. Am. 24:1047 (1995); Agozzino, F. et al. Ital. Heart J. 3:686 (2002)]. The most valuable predictor of mortality in the scenery of ALI is a serum bilirubin level >3 times the upper limit of normal [Bjornsson, E. Clin. Pharmacol. Ther. 79:521 (2006); Reuben, A. Hepatology, 39:574 (2004)].

The acute cholestatic injury frequently resembles extrahepatic obstructive jaundice. Cholestatic injury that may be also treated by the invention, is characteristically predictable by predominant elevations in alkaline phosphatase and bilirubin. Medications that have been associated with acute cholestatic injury include amoxicillin-clavulanate, chlorpromazine, nafcillin, trimethoprim-sulfamethoxazole, rifampin, erythromycin estolate, captopril, estradiol, and rarely, amiodarone [Stieger, B. et al. Gastroenterology, 118:422 (2000]. The disease is largely subclinical and the most common symptoms are pruritus and jaundice. Serum aminotransferases are only hardly elevated (usually less than eightfold). Comparing the overall prognosis for purely cholestatic injury is better than for hepatocellular injury.

On liver biopsy 4 types of histology can be found in the cholestatic form: Canalicular, Hepatocanalicular, Ductopenic cholestasis and Sclerosing cholangitis [Ostapowicz, G. et al. Ann. Intern. Med. 137:947 (2002); Zimmerman, H. J. Schiffs Diseases of the Liver, Schiff, E. Sorrell, M. Maddrey, W. (Eds), Lippincott-Raven, Philadelphia, Pa.: p. 973 (1999); Speeg, K. V. and Bay, M. K. Gastroenterol. Clin. North. Am. 24:1047 (1995); Stieger, B. et al. Gastroenterology, 118:422 (2000); Zimmerman, H. J. and Lewis, J. H. Gastroenterol. Clin. North. Am. 24:1027 (1995)].

Mixed patterns of injury are frequent, and show elevations in both aminotransferases and alkaline phosphatase. Patients with hepatotoxicity due to phenyloin can present mixed pattern, and may be at increased risk to develop chronic liver disease compared with other forms of hepatotoxicity [Andrade, R. J. et al. Hepatology 44:1581 (2006)].

Drug-induced acute steatosis (fatty degeneration) that may be also treated by the methods, kits and combined compositions of the invention, is uncommon and can lead to clinical features analogous to Reye's syndrome or acute fatty liver of pregnancy. In this pattern jaundice is often mild and serum aminotransferases are lower than that seen in cytotoxic injury. Even though biochemical features generally do not appear as severe as those seen in hepatocellular disease, the illness can be severe and the prognosis unfortunate with high mortality [Huang, Y. L. et al. J. Am. Acad. Dermatol. 49:316 (2003)]. This is especially true of steatohepatitis related to Reye's syndrome, high-dose intravenous tetracycline, and amiodarone. Microvesicular steatosis developed with the use of numerous drugs such as cocaine, valproic acid, and the antiretroviral agents zidovudine (AZT), stavudine, and didanosine (ddI) [Zimmerman, H. J. and Lewis, J. H. Gastroenterol. Clin. North. Am. 24:1027 (1995); Miller, K. D. et al. Ann. Intern. Med. 133:192 (2000); Koch, R. O. et al. Wien. Klin. Wochenschr. 115:135 (2003)]. Herbal remedies are being increasingly identified as causes of steatosis and other forms of injury [Chitturi, S, and Farrell, G. C. J. Gastroenterol. Hepatol. 15:1093 (2000); Stedman, C. Semin. Liver Dis. 22:195 (2002)].

In another embodiment, the beta-glycolipid used for the method of the invention may be selected from the group consisting of a glucosylceramide, a monosaccharide ceramide, a galatosylceremide, a lactosyl-ceramide, a gal-gal-glucosyl-ceramide, GM2 ganglioside, GM3 ganglioside, globoside or any synthetic or natural β-glycolipid or any derivative or combination thereof, specifically, glucosylceramide (GC).

Still further, the tocopherol (vitamin E), tocotrienol or any derivatives thereof used by the method of the invention may be selected from the group consisting of: alpha-tocopherol, beta-tocopherol, gamma-tocopherol, delta-tocopherol, alpha-tocotrienol, beta-tocotrienol, gamma-tocotrienol, delta-tocotrienol and any combination thereof, specifically, alpha-tocopherol.

Thus, according to one specific embodiment, the method of the invention uses a combination of glucosylceramide (GC) with alpha-tocopherol. As clearly demonstrated by the following examples, the combination of both GC and Vitamin E resulted in a synergistic combination having a significant ameliorating effect on chronic and acute hepatotoxic manifestations of acetaminophen. Surprisingly, the present invention now show that reduction of liver injury due to acetaminophen ingestion, may be achieved by the use of combination of glucosylceramide (GC) and vitamin E that promote CD3+NK1.1+ (NKT lymphocytes) and the glutathione system. The results show a small increase in hepatic NKT cells after the treatment of GC. However, after the pretreatment of vitamin E, the increase in NKT cells became significant, and after the pretreatment of the combination of GC and vitamin E, that increase in hepatic NKT cells, remained significant. A possible mechanism for that effect may involve the high antioxidant activity of vitamin E and the lower toxicity of the liver achieved by the activity of GC. These two effects may involve high activity of hepatic NKT cells which are known to be up-regulated in acetaminophen toxicity, which are more beneficial in an antioxidant environment.

According to another embodiment, the GC-alpha-tocopherol combination of the invention may be administered before the administration of acetaminophen to the treated subject, thereby preventing and reducing any hepatotoxic effects caused thereby.

In another embodiment, the GC-alpha-tocopherol, specifically, GC and vitamin E, combination of the invention may be administered simultaneously with the administration of acetaminophen to the treated subject. Such simultaneous administration may also prevent, reduce or ameliorate any hepatotoxic effect that may be caused by acetaminophen.

It should be appreciated that such simultaneous administration may be performed by administering a combined composition comprising GC, alpha-tocopherol and acetaminophen.

In yet another embodiment, the GC-alpha-tocopherol combination of the invention may be administered after the administration of acetaminophen to the treated subject.

According to one embodiment, the GC-alpha-tocopherol combination of the invention may be administered within forty-eight to ninety-six hours of the administration of acetaminophen to said subject, or at any time point before or after administration of the toxic drug, or at any time point before or after any type of liver insults due to infectious, metabolic, toxic, immune, perfusion or blood flow reasons occurred.

The combined beta-glycolipid and vitamin E compositions can be administered from one or more times per day to one or more times per week, including once every other day. The combined beta-glycolipid and vitamin E compositions can be administered, e.g., for about 1 to 30, 5 to 14 days or longer. More specifically, the composition may be administered for about 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30 days. The skilled artisan will appreciate that certain factors may influence the dosage and timing required to effectively treat a subject, including but not limited to the severity of the disease or disorder, previous treatments, the general health and/or age of the subject, and other diseases present.

Moreover, treatment of a subject with a therapeutically effective amount of the combined compounds can include a single treatment or, can include a series of treatments.

By “patient” or “subject in need” it is meant any mammal who may be affected by the above-mentioned conditions, and to whom the treatment and diagnosis methods herein described is desired, including human, bovine, equine, canine, murine and feline subjects. Preferably said patient is a human. Administering of the drug combination to the patient includes both self-administration and administration to the patient by another person.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician.

According to a specific embodiment, a therapeutically effective amount used by the combined compositions, methods and kits of the invention, may comprise between about 0.0001 to 100 mg per kg, specifically, 1.5 mg per kg of body weight β-glucosylceramide, in another specific embodiment, 0.5 mg per kg of body weight β-glucosylceramide and between about 0.0001 to 100 mg per kg, specifically, 2.5 gr per kg of body weight vitamin E.

The terms “treat, treating, treatment” as used herein and in the claims mean ameliorating one or more clinical indicia of disease activity in a patient having a pathologic condition that may be ameliorated, delayed or prevented by the combined composition of the invention.

“Treatment” refers to therapeutic treatment. Those in need of treatment are mammalian subjects suffering from any pathologic condition that may be ameliorated, delayed or prevented by the combined composition of the invention. By “patient” or “subject in need” is meant any mammal for which administration of the combined composition of the invention, is desired, in order to prevent, overcome or slow down such infliction. It should be noted that the protective and therapeutic effect of the combined composition of the invention is clearly demonstrated in the Examples, showing clear alleviation of acetaminophen-induced liver damage as exhibited by reduction of ALT and AST levels.

It should be appreciated that the terms “inhibition”, “moderation”, “reduction” or “attenuation” as referred to herein, relate to the retardation, restraining or reduction of a process (for example, a process that leads to elevation in AST or ALT levels) by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

To provide a “preventive treatment” or “prophylactic treatment” is acting in a protective manner, to defend against or prevent something, especially a condition or disease.

“Mammal” or “mammalian” for purposes of treatment refers to any animal classified as a mammal including, human, research animals, domestic and farm animals, and zoo, sports, or pet animals, such as dogs, horses, cats, cows, etc. In a particular embodiment said mammalian subject is a human subject.

According to another embodiment, the combination of the invention or any composition or kit thereof may be administered by oral, intravenous, intramuscular, subcutaneous, intraperitoneal, perenteral, transdermal, intravaginal, intranasal, mucosal, sublingual, topical, rectal or subcutaneous administration, or any combination thereof.

According to a specific embodiment, the composition of the invention is particularly suitable for oral or mucosal administration.

The usefulness of an oral formulation requires that the active agent or combinations of the invention be bio-available.

Bioavailability of orally administered drugs can be affected by a number of factors, such as drug absorption throughout the gastrointestinal tract, stability of the drug in the gastrointestinal tract, and the first pass effect. Thus, effective oral delivery of an active agent or combination requires that the active agent have sufficient stability in the stomach and intestinal lumen to pass through the intestinal wall. Many drugs, however, tend to degrade quickly in the intestinal tract or have poor absorption in the intestinal tract so that oral administration is not an effective method for administering the drug.

More specifically, the composition of the invention may be suitable for mucosal administration, for example, pulmonary, buccal, nasal, intranasal, sublingual, rectal, vaginal administration and any combination thereof.

Pharmaceutical compositions suitable for oral administration are typically solid dosage form (e.g., tablets) or liquid preparations (e.g., solutions, suspensions, or elixirs).

Solid dosage forms are desirable for ease of determining and administering dosage of active ingredient, and ease of administration, particularly administration by the subject at home.

Liquid dosage forms also allow subjects to easily take the required dose of active ingredient. Liquid preparations can be prepared as a drink, or to be administered, for example, by a nasal-gastric tube (NG tube). Liquid oral pharmaceutical compositions generally require a suitable solvent or carrier system in which to dissolve or disperse the active agent, thus enabling the composition to be administered to a subject. A suitable solvent system is compatible with the active agent and non-toxic to the subject. Typically, liquid oral formulations use a water-based solvent.

The oral compositions of the invention can also optionally be formulated to reduce or avoid the degradation, decomposition, or deactivation of the active agents by the gastrointestinal system, e.g., by gastric fluid in the stomach. For example, the compositions can optionally be formulated to pass through the stomach unaltered and to dissolve in the intestines, i.e., enteric coated compositions.

A further aspect of the invention relates to the use of a therapeutically effective amount of a combination of at least one natural or synthetic beta-glycolipid and at least one tocopherol (vitamin E), tocotrienol or any derivatives thereof and optionally further comprises at least one additional therapeutic agent, in the preparation of a medicament for treating, preventing, ameliorating, reducing or delaying the onset of acute or chronic toxic effect of an analgesic or an antipyretic drug or any type of liver insult selected from infectious metabolic, toxic, immune, or perfusion or blood flow related hepatic injury.

According to one embodiment of said aspect, the use of the combined composition of the invention is particularly applicable for acute or chronic toxic effect such as drug induced liver injury (DILI), drug-induced acute steatosis, cytotoxic hepatocellular injury, acute liver failure (ALF), reperfusion injury, ischemic liver disease and acute cholestatic injury.

In yet another embodiment, the use of the invention relates to a combined composition comprising at least one beta-glycolipid that may be selected from the group consisting of a glucosylceramide, a monosaccharide ceramide, a galatosylceremide, a lactosyl-ceramide, a gal-gal-glucosyl-ceramide, GM2 ganglioside, GM3 ganglioside, globoside or any synthetic or natural β-glycolipid or any derivative or combination thereof, specifically, GC, and at least one tocopherol (vitamin E), tocotrienol or any derivatives thereof selected from the group consisting of: alpha-tocopherol, beta-tocopherol, gamma-tocopherol, delta-tocopherol, alpha-tocotrienol, beta-tocotrienol, gamma-tocotrienol, delta-tocotrienol and any combination thereof, specifically, alpha-tocopherol.

According to one specific embodiment, the invention provides the use of a combined composition comprising glucosylceramide (GC) and alpha-tocopherol.

According to another embodiment, the use according to the invention of the combined composition of the invention is particularly applicable for acute or chronic toxic effect caused by an analgesic drug, such as acetaminophen (paracetamol).

As indicated herein, the invention provides the use of a therapeutically effective amount of a combination of at least one natural or synthetic beta-glycolipid and at least one tocopherol (vitamin E), tocotrienol or any derivatives thereof, in the preparation of a medicament for treating, preventing, ameliorating, reducing or delaying the onset of acute or chronic toxic effect of an analgesic or an antipyretic drug.

The present invention therefore particularly relates to additive and synergistic combinations of at least one beta-glycolipid and at least one vitamin E, preferably, the specific combination of GC and alpha-tocopherol.

Those additive and synergistic combinations are useful in treating subjects suffering from acute or chronic toxic effect of acetaminophen, for example, drug induced liver injury (DILI). The synergistic and additive compositions of the invention may also be used for the treatment of subjects presenting with symptoms or signs of such disorders.

By synergic combination is meant that the effect of both beta-glycolipid and vitamin E is greater than the sum of the therapeutic effects of administration of any of these compounds separately, as a sole treatment.

The invention further provides a pharmaceutical unit dosage form comprising at least one natural or synthetic beta-glycolipid and at least one tocopherol (vitamin E), tocotrienol or any derivatives thereof and optionally further comprises at least one additional therapeutic agent, and a pharmaceutically acceptable carrier or diluent.

As indicated above, the combined beta-glycolipids and vitamin E described herein can be incorporated into a pharmaceutical composition suitable for oral or mucosal administration, e.g., by ingestion, inhalation, or absorption, e.g., via nasal, intranasal, pulmonary, buccal, sublingual, rectal, or vaginal administration. Such compositions can include an inert diluent or an edible carrier. For the purpose of oral therapeutic administration, the active compound (e.g., combination of vitamin E and a beta-glucosylceramide (GC) can be incorporated with recipients and used in solid or liquid (including gel) form. Oral compositions can also be prepared using an excipient. Pharmaceutically compatible binding agents, and/or adjuvant materials can be included as part of the composition. Oral dosage forms comprising combined beta-glycolipid and vitamin E are provided, wherein the dosage forms, upon oral administration, provide a therapeutically effective blood level of the combined beta-glycolipid and vitamin E to a subject. Also provided are mucosal dosage forms comprising said combination wherein the dosage forms, upon mucosal administration, provide a therapeutically effective blood level of the combined beta-glycolipid and vitamin E to a subject. For the purpose of mucosal therapeutic administration, the active combined compounds (e.g., beta-glucosylceramide with vitamin E) can be incorporated with excipients or carriers suitable for administration by inhalation or absorption, e.g., via nasal sprays or drops, or rectal or vaginal suppositories.

Solid oral dosage forms include, but are not limited to, tablets (e.g., chewable tablets), capsules, caplets, powders, pellets, and granules, powder in a sachet, enteric coated tablets, enteric coated beads, and enteric coated soft gel capsules. Also included are multi-layered tablets, wherein different layers can contain different drugs. Solid dosage forms also include powders, pellets and granules that are encapsulated. The powders, pellets, and granules can be coated, e.g., with a suitable polymer or a conventional coating material to achieve, for example, greater stability in the gastrointestinal tract, or to achieve a desired rate of release.

In addition, a capsule comprising the powder, pellets or granules can be further coated. A tablet or caplet can be scored to facilitate division for ease in adjusting dosage as needed.

The dosage forms of the present invention can be unit dosage forms wherein the dosage form is intended to deliver one therapeutic dose per administration, e.g., one tablet is equal to one dose. Such dosage forms can be prepared by methods of pharmacy well known to those skilled in the art. Typical oral dosage forms can be prepared by combining the active ingredients in an intimate admixture with at least one excipient according to conventional pharmaceutical compounding techniques. Excipients can take a wide variety of forms depending on the form of preparation desired for administration. For example, excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents. Examples of excipients suitable for use in oral liquid dosage forms include, but are not limited to, water, glycols, oils, alcohols, flavoring agents, preservatives, and coloring agents. Tablets and capsules represent convenient pharmaceutical compositions and oral dosage forms, in which case solid excipients are employed. If desired, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

As one example, a tablet can be prepared by compression or by molding. Compressed tablets can be prepared, e.g., by compressing, in a suitable machine, the active ingredients (e.g., combined beta-glycolipid and vitamin E) in a free-flowing form such as powder or granules, optionally mixed with an excipient. Molded tablets can be made, e.g., by molding, in a suitable machine, a mixture of the powdered combined beta-glycolipid and vitamin E compound moistened, e.g., with no inert liquid diluent.

Excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gum tragacanth or gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidinones, methyl cellulose, pro-gelatinized starch, hydroxypropyl methyl cellulose, microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions and dosage forms of the invention is typically present in from about 50 to about 99 weight percent of the pharmaceutical composition or dosage form.

Disintegrants can be used in the pharmaceutical compositions and oral or mucosal dosage forms of the invention to provide tablets that disintegrate when exposed to an aqueous environment. Tablets containing too much disintegrant might disintegrate in storage, while those containing too little might not disintegrate at a desired rate or under desired conditions.

Thus, a sufficient amount of disintegrant that is neither too much nor too little to detrimentally alter the release of the active ingredients should be used to form the pharmaceutical compositions and solid oral dosage forms described herein. The amount of disintegrant used varies based upon the type of formulation, and is readily discernible to those of ordinary skill in the art. Typically, pharmaceutical compositions and dosage forms comprise from about 0.5 to about 15 weight percent of disintegrant, preferably from about 1 to about 5 weight percent of disintegrant.

Disintegrants that can be used in pharmaceutical compositions and oral or mucosal dosage forms of the invention include, but are not limited to, agar-agar, alginic acid, calcium carbonate, Primogel, microcrystalline cellulose, croscarmellose sodium, crospovidone, polacrilin potassium, sodium starch glycolate, corn, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate or Sterotes, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof.

The pharmaceutical compositions and oral or mucosal dosage forms can further comprise one or more compounds that reduce the rate by which an active ingredient will decompose. Thus the oral dosage forms described herein can be processed into an immediate release or a sustained release dosage form. Immediate release dosage forms may release the combined beta-glycolipid and vitamin E in a fairly short time, for example, within a few minutes to within a few hours. Sustained release dosage forms may release the combined beta-glycolipid and vitamin E over a period of several hours, for example, up to 24 hours or longer, if desired. In either case, the delivery can be controlled to be substantially at a certain predetermined rate over the period of delivery. In some embodiments, the solid oral dosage forms can be coated with a polymeric or other known coating material(s) to achieve, for example, greater stability on the shelf or in the gastrointestinal tract, or to achieve control over drug release. Such coating techniques and materials used therein are well-known in the art. Such compounds, which are referred to herein as “stabilizers,” include, but are not limited to, antioxidants such as ascorbic acid and salt buffers. For example, cellulose acetate phthalate, polyvinyl acetate phthalate, hydroxypropylmethyl cellulose phthalate, methacrylic acid-methacrylic acid ester copolymers, cellulose acetate trimellitate, carboxymethylethyl cellulose, and so hydroxypropylmethyl cellulose acetate succinate, among others, can be used to achieve enteric coating. Mixtures of waxes, shellac, rein, ethyl cellulose, acrylic resins, cellulose acetate, silicone elastomers can be used to achieve sustained release coating.

Liquids for oral or mucosal administration represent another convenient dosage form, in which case a solvent can be employed. In some embodiments, the solvent is a buffered liquid such as phosphate buffered saline (PBS). Liquid oral dosage forms can be prepared by combining the active ingredient in a suitable solvent to form a solution, suspension, syrup, or elixir of the active ingredient in the liquid. The solutions, suspensions, syrups, and elixirs may optionally comprise other additives including, but not limited to, glycerin, sorbitol, propylene glycol, sugars or other sweeteners, flavoring agents, and stabilizers. Flavoring agents can include, but are not limited to peppermint, methyl salicylate, or orange flavoring. Sweeteners can include sugars, aspartame, saccharin, sodium cyclamate and xylitol.

For administration by inhalation, the mucosal combined beta-glycolipid and vitamin E compounds can be delivered in the form of an aerosol spray from pressured container or dispenser which contains a suitable propellant, e.g., a gas such as carbon dioxide, or a nebulizer.

Systemic administration can also be by transmucosal means. For transmucosal administration, penetrants appropriate to the barrier to be permeated are used in the formulation. Such penetrants are generally known in the art, and include, for example, for transmucosal administration, detergents, bile salts, and fusidic acid derivatives.

Transmucosal administration can be accomplished through the use of nasal drops or sprays, or rectal or vaginal suppositories.

Dosage, toxicity and therapeutic efficacy of such combined beta-glycolipid and vitamin E compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between so toxic and therapeutic effects is the therapeutic index and it can be expressed as the ratio LD50/ED50. Compositions which exhibit high therapeutic indices are preferred.

The combined compounds of the present invention are generally administered in the form of a pharmaceutical composition comprising both compounds of this invention together with a pharmaceutically acceptable carrier or diluent. Thus, the compounds used by this invention can be administered either individually in a kit or together in any conventional oral or mucosal dosage form.

More particularly, since the present invention relates to the treatment of diseases and conditions with a combination of active ingredients which may be administered separately, the invention also relates as a further aspect, to combining separate pharmaceutical compositions in kit form. The kit includes at least two separate pharmaceutical compositions: beta-glycolipid and vitamin E.

Thus, another aspect of the invention relates to a kit for achieving a therapeutic effect in a subject suffering from acute or chronic toxic effect of an analgesic or an antipyretic drug or any type of liver insult selected from infectious metabolic, toxic, immune, or perfusion or blood flow related hepatic injury. According to one embodiment, the kit of the invention comprises:

(a) at least one natural or synthetic beta-glycolipid or a pharmaceutically acceptable derivative thereof and a pharmaceutically acceptable carrier or diluent, optionally in a first unit dosage form;
(b) at least one tocopherol (vitamin E), tocotrienol or any derivatives thereof, and a pharmaceutically acceptable carrier or diluent, optionally, in a second unit dosage form; and
(c) container means for containing said first and second dosage forms.

More specifically, the kit includes container means for containing both separate compositions; such as a divided bottle or a divided foil packet however, the separate compositions may also be contained within a single, undivided container. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

According to one embodiment, the kit of the invention may be particularly applicable for treating acute or chronic toxic effect caused by a drug. Examples for such toxic effect include for example, drug induced liver injury (DILI), drug-induced acute steatosis, cytotoxic hepatocellular injury, acute liver failure (ALF), reperfusion injury, ischemic liver disease and acute cholestatic injury.

According to one embodiment, the beta-glycolipid comprised within the kit of the invention may be selected from the group consisting of a glucosylceramide, a monosaccharide ceramide, a galatosylceremide, a lactosyl-ceramide, a gal-gal-glucosyl-ceramide, GM2 ganglioside, GM3 ganglioside, globoside or any synthetic or natural β-glycolipid or any derivative or combination thereof, specifically, glucosylceramide (GC).

In yet another embodiment, the tocopherol (vitamin E), tocotrienol or any derivatives thereof comprised within the kit of the invention may be selected from the group consisting of: alpha-tocopherol, beta-tocopherol, gamma-tocopherol, delta-tocopherol, alpha-tocotrienol, beta-tocotrienol, gamma-tocotrienol, delta-tocotrienol and any combination thereof, specifically, alpha-tocopherol.

Thus, according to one specific embodiment, the kit of the invention comprises glucosylceramide (GC), optionally, in a first dosage unit form, and alpha-tocopherol, specifically, vitamin E, optionally, in a second dosage unit form.

According to another embodiment, the kit of the invention is intended for achieving a therapeutic effect in a subject suffering from acute or chronic toxic effect of an analgesic or antipyretic drug that may be an inducer or inhibitor of Cytochrom P-450. Such Cytochrom P-450 may be selected from the group consisting of: Acetaminophen, Phenobarbital, Phenyloin, Carbamazepine, Primidone, Ethanol, Glucocorticoids, Rifampin, Griseofulvin, Quinine, Omeprazole, Amiodarone, Cimetidine, Erythromycin, Grape fruit, Isoniazid, Ketoconazole, Metronidazole, Sulfonamides, Chlorpromazine, phenylbutazone, halogenated anesthetic agents, sulindac, Dapsone, INH, halothane, amoxicillin-clavulanic acid, phenobarbital, Para-amino salicylate, Clofibrate, Procainamide, Gold salts, propylthiouracil, chloramphenicol, nitrofurantoin, methoxyflurane, penicillamine, paraquat, Tetracycline, Contraceptive and anabolic steroids, rifampin, Aspirin and Sodium valproate.

According to another specific embodiment, the kit of the invention is intended for achieving a therapeutic effect in a subject suffering from acute or chronic toxic effect of an analgesic drug such as acetaminophen (paracetamol).

Achieving a therapeutic effect is meant for example, where the kit is intended for the treatment of a specific disorder, the therapeutic effect may be, for example, slowing the progression of the treated condition.

It should be appreciated that both components of the kit, the beta-glycolipid in the first dosage form and the vitamin E in the second dosage form may be administered simultaneously.

Alternatively, said first compound or dosage form and said second compound or dosage form are administered sequentially in either order.

Still further, the invention provides a method of treating, preventing, ameliorating, reducing or delaying the onset of acute or chronic toxic effect of analgesic or antipyretic drug in a subject in need thereof. The method the invention comprises the step of administering to the treated subject a therapeutically effective amount of a first and a second unit dosage forms comprised in a kit according to the invention, before, simultaneously with, after or any combination thereof, administration of the drug to the treated subject. Specifically, the kit of the invention is administered by the method of the invention to a subject suffering of acute or chronic toxic effect caused by acetaminophen.

The present invention demonstrates a combined composition ameliorating the toxic effects caused by over-dose of acetaminophen, and therefore further provides a method for increasing the maximum amount of acetaminophen that can be administered to a subject without exhibiting acetaminophen toxicity. Such method according to the invention comprises administering an acetaminophen toxicity inhibiting amount of a combination of at least one natural or synthetic beta-glycolipid and at least one tocopherol (vitamin E), tocotrienol or any derivatives thereof, or any composition or any kit thereof, before, simultaneously with, after or any combination thereof, administration of said acetaminophen to said subject.

More particularly, reduction of acetaminophen toxicity is the amelioration, reversal, reduction, or any combination thereof of acetaminophen toxicity effects in an individual after acetaminophen administration that results in either chronic toxicity or acute toxicity, including acetaminophen overdose. Increasing the amount of acetaminophen that can be administered to a mammal without producing acetaminophen toxicity enables those who have increased sensitivity to acetaminophen to tolerate normally non-toxic or even higher dosages of acetaminophen and allows those individuals to avail themselves of the full therapeutic effects of acetaminophen. Increasing the amount of acetaminophen that can be administered to an individual also permits those having normal sensitivity to acetaminophen to take increased dosages of acetaminophen, which would ordinarily produce toxic effects, without those toxic effects.

A further aspect of the invention relates to a method of inducing at least one T regulatory cell in a subject in need thereof, specifically, a subject suffering of acute or chronic effect of acetaminophen. The method of the invention comprises the step of administering to said subject a therapeutically effective amount of at least one of:

(a) a combination of at least one natural or synthetic beta-glycolipid and at least one tocopherol (vitamin E), tocotrienol or any derivatives thereof;
(b) an immune-cell treated with any one of (a) or with any composition comprising the same;
(c) an immune-cell obtained from a subject treated with any one of (a), (b) or any combination or mixtures thereof or any composition comprising the same; and
(d) a composition comprising any one of (a), (b), (c) or any combinations or mixtures thereof, said composition optionally further comprises at least one pharmaceutically acceptable carrier, diluent, excipient and/or additive.

The combined composition of the invention may also exhibit a stimulatory effect on immune-related cells. An immune-related cell may be an APC (such as DC), Treg cell or any other cell associated directly or indirectly with the immune system. Such cells include but are not limited to platelets, macrophages, any type of B cell, T cell (including double negative cells), and any type of non-professional antigen presenting cell, adipocytes, endothelial cell, any type of cell that is part of an organ, specifically, an organ connected to the treated immune-related disorder and any type of cell having regulatory enhancing or suppressing properties. More particularly, the combined composition of the invention may demonstrate activation of immune-related cells such as specific T regulatory cells for example, CD4+LAP+, adipocytes and Antigen Presenting Cells (APC), such as DC, thereby ameliorating liver damage. Therefore, according to one embodiment, the composition of the invention may be used for inducing at least one of T regulatory (Treg) cells, any cell having regulatory properties, either suppressive or inductive, adipocyte and Antigen Presenting Cells (APC) in a subject suffering from acute or chronic toxic effect of acetaminophen. More specifically, immune-related cells induced by the composition of the invention may be any T regulatory cell, for example any one of CD4+LAP+T-reg cells, CD4+CD25 T-reg cells, CD8+CD25 T-reg cells, FoxP3+CD4 T-reg cells, CD25 High T-reg cells, CD127 MFI T-reg cells, CD28 MFI T-reg cells, CTLA4-T-reg cells and HLA-DR T-reg cells. According to one specific embodiment, the combined composition of the invention induces CD4+LAP+T-reg cells.

Unless otherwise defined, all technical and scientific terms used herein have the same meaning as commonly understood by one of ordinary skill in the art to which this invention belongs. Although methods and materials similar or equivalent to those described herein can no be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

### Animals

Male C57B1/6 mice (11-12 weeks old) were obtained from Harlan Laboratories (Jerusalem, Israel) and maintained in the Animal Core of the Hadassah-Hebrew University Medical School.

All mice were administered standard laboratory chow and water ad libitum and kept in a 12-hour light/dark cycle. The animal experiments were carried out according to the guidelines of the Hebrew University-Hadassah Institutional Committee for Care and Use of Laboratory Animals and with the committee's approval.

### Preparation of Glycolipids

β-glucosylceramide (GC) was purchased from Avanti Polar Lipids (Alabaster, Ala., USA). Glycolipids (3.3 mg/ml) were dissolved in a mixture of 30% Cremophor EL (Sigma, Rehovot, Israel) and ethanol (1:1, C:E) in PBS.

### Vitamins

Vitamin E: 5gr α-Tocopherol, keep in dark, 4° C., purchased from Sigma (cat. #258024), 50 mg/1000/mice were administered by gavage.
Vitamin C: 1 mg L-Ascorbic acid, keep in dark, 4° C., purchased from Sigma (cat. #47863), 50 mg/100 μl/mice were administered by gavage.
Vitamin B6: 1gr Pyridoxal hydrochloride, keep in dark, 4° C., purchased from Sigma (cat. # P9130), 50 mg/100 μl/mice were administered by gavage.

### Acetaminophen

Acetaminophen Sigma (cat. # A7085), was dissolved in a mixture of 30% Cremophor EL (Sigma, Rehovot, Israel) and ethanol (1:1) in DDW (Di distilled water).

### AST and ALT Levels as Parameters of Liver Injury

Mice were tested for serum Alanine transaminase (ALT) and Aspartate aminotransferase (AST) at 24 hours after acetaminophen administration. Serum AST and ALT levels were measured by an automatic analyzer.

### Isolation of Splenocytes and Hepatic Lymphocytes

Splenocytes and hepatic lymphocytes were isolated as previously described [Margalit, M. et al. Am. J. Physiol. Gastrointest. Liver Physiol: 289 917-25 (2000)]. Livers and spleens were maintained in RPMI-1640 supplemented with fetal bovine serum. Spleens were crushed through a 70-μm nylon cell strainer and centrifuged (1250 rpm for 7 min) to remove debris. Red blood cells were lysed with 1 mL cold 155 mM ammonium chloride lysis buffer and immediately centrifuged (1250 rpm for 3 min). Splenocytes were washed and resuspended in 1 mL RPMI plus fetal bovine serum. The connective tissue remnants were removed. Cell viability was determined by trypan blue staining and was greater than 90%.

For intrahepatic lymphocytes, livers were first crushed through a stainless mesh (size 60, Sigma) and cell suspension was placed in a 50-mL tube for 5 min to pellet the cell debris. Ten milliliters of Lymphoprep (Ficoll, Axis-Shield PoC AS, Oslo, Norway) was slowly placed under the same volume of cell suspension in 50-mL tubes. The tubes were then centrifuged at 1800 rpm for 18 min. Cells at the interface were collected and transferred to new tubes, which were then centrifuged at 1800 rpm for 10 min, to obtain a cell pellet depleted of hepatocytes in a final volume of 250 μl. Approximately 1×10⁶ cells/mouse liver were recovered.

### Flow Cytometry for Lymphocyte Subsets

Flow cytometry was performed following lymphocyte isolation using 1×10⁶ lymphocytes in 100 μl PBS. To determine the percentage of NKT lymphocytes, Pacific Blue anti-mouse CD3, PE anti-mouse NK1.1 and APC-anti mouse TCR-β antibodies were used (eBioscience, USA). Pacigic Blue anti mouse CD4, FITC-anti mouse CD8 subsets were also used ((eBioscience, USA). For intracellular staining we used PE-anti mouse CD25 and PE-Cy7 anti mouse FOXp3 (eBioscience, USA). Cells were incubated for 30 min at 4° C. in the dark, and washed and resuspended in 200 μl PBS. Analytical cell sorting was performed on 1×10⁴ cells from each group using a fluorescence-activated cell sorter (FACSTAR plus, Becton Dickinson). Only live cells were counted and unstained cells served as controls for background fluorescence. Gates were set on forward- and side-scatters to exclude dead cells and red blood cells. Data were analyzed using either the Consort 30 two-color contour plot program (Becton Dickinson, Oxnard, Calif.) or the CELLQuest 25 program.

### Determination of Cellular Glutathione Activity

The activity of reduced glutathione was assayed in the sera of treated mice using the QuantiChrom™ Glutathione Assay Kit (DIGT-250), according to the manufacturer's instructions.

### Statistical Analysis

The comparison of two independent groups was performed using the Student's t test. The association between two variables was assessed by calculating the Pearson and the Spearman correlation coefficients. All tests applied were two-tailed, and a p value of 0.05 or less was considered statistically significant.

### Example 1

### Amelioration of Acetaminophen-Induced Liver Injury by GC

Beta glycolipides were previously shown by the present inventors as having immunomodulatory effects. More specifically, the inventors demonstrated that β-glycolipides and particularly, GC promote LAP⁺ regulatory T lymphocytes. The inventors therefore next examined whether such effect may also enhance the glutathione system and thereby ameliorate hepatic injury induced by drugs such as acetaminophen. The effect of GC on acetaminophen-induced liver injury was evaluated by examination of liver enzyme (ALT and AST). Three groups of mice, 15 animals per group, were studied. Mice of all groups were fasted for eight hours before oral administration of acetaminophen and for additional four hours after administration. Mice in all groups were administered orally (PO) with 10 mg/330 μl acetaminophen (in C:E gavage administration). In addition, mice were administered with 100 μg β-glucosylceramide (β-GC, in 30 μl of C:E) two hours before administration of acetaminophen (Group B) and every two hours after acetaminophen administration (Group C), Mice in control group A were treated only with oral PBS.

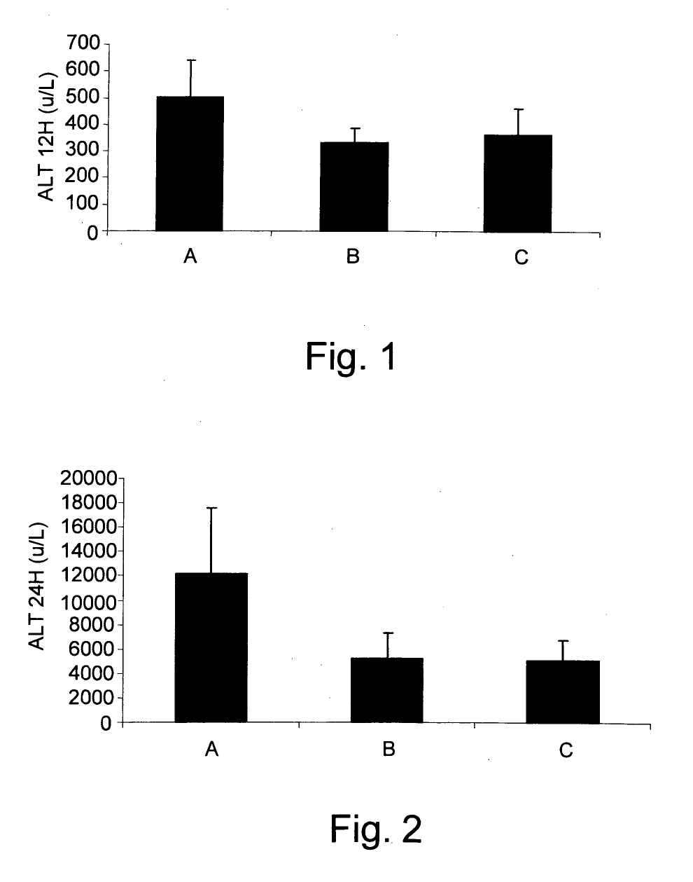

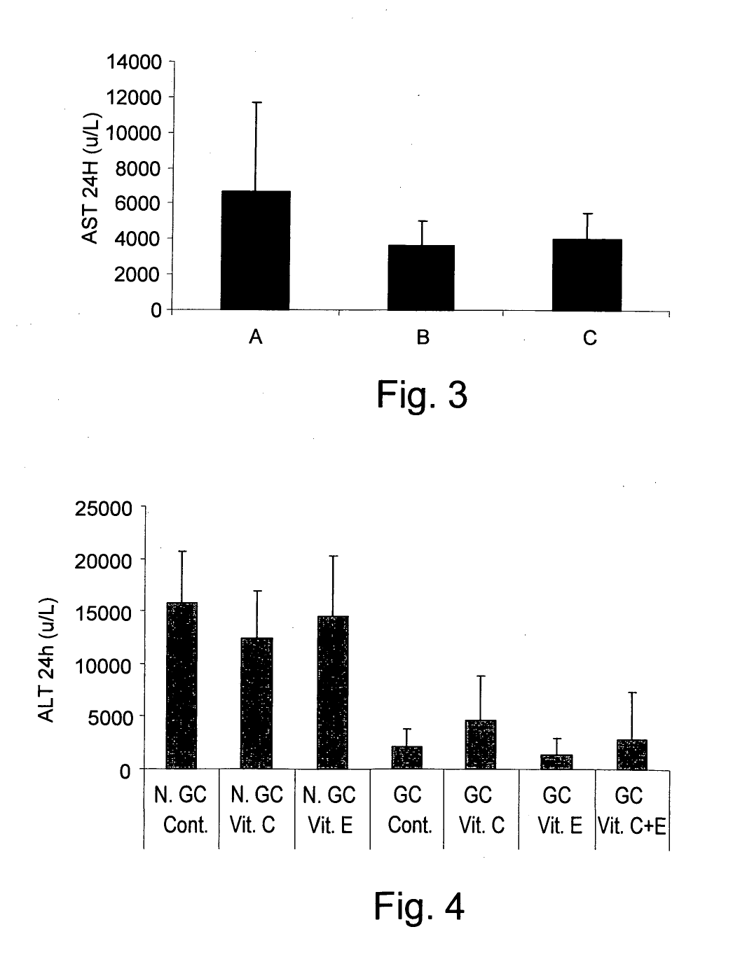

As demonstrated by FIG. 1, examination of ALT levels after twelve hours indicated a highly significant statistical amelioration of liver damage shown by lower ALT levels for group B and C as compared with the control group A (332 and 364 Vs 505 for group B, C and A, respectively p<0.001). Samples taken after twenty four hours demonstrated even more significant difference in ALT and AST levels (FIGS. 2 and 3, respectively) for group B and C as compared with group A (ALT, 5222, 5034 Vs 12150 p<0.001. AST 3650, 4029 Vs 6634 p<0.04). It should be noted that gavage of GC before induction of liver damage enhanced the beneficial effect of GC.

### Example 2

### Synergistic Combination of GC and Vitamin E Significantly Alleviates Acetaminophen-Induced Liver Damage

Vitamin E levels have been shown to be decreased in chronic liver diseases of different etiology. Vitamin E, being a potent antioxidant was proposed for nonalcoholic fatty liver disease (NAFLD), the most frequent hepatic lesion in western countries which can progress to nonalcoholic steatohepatitis and cirrhosis due to the production of large amounts of oxidative stress products. Encouraged by the ameliorating effect of GC on acetaminophen-induced liver damage, the inventors next examined the potential of combining GC with several vitamins, including vitamin C, D, B6 and E, in alleviating acetaminophen-induced liver damage as reflected by examination of ALT and AST.

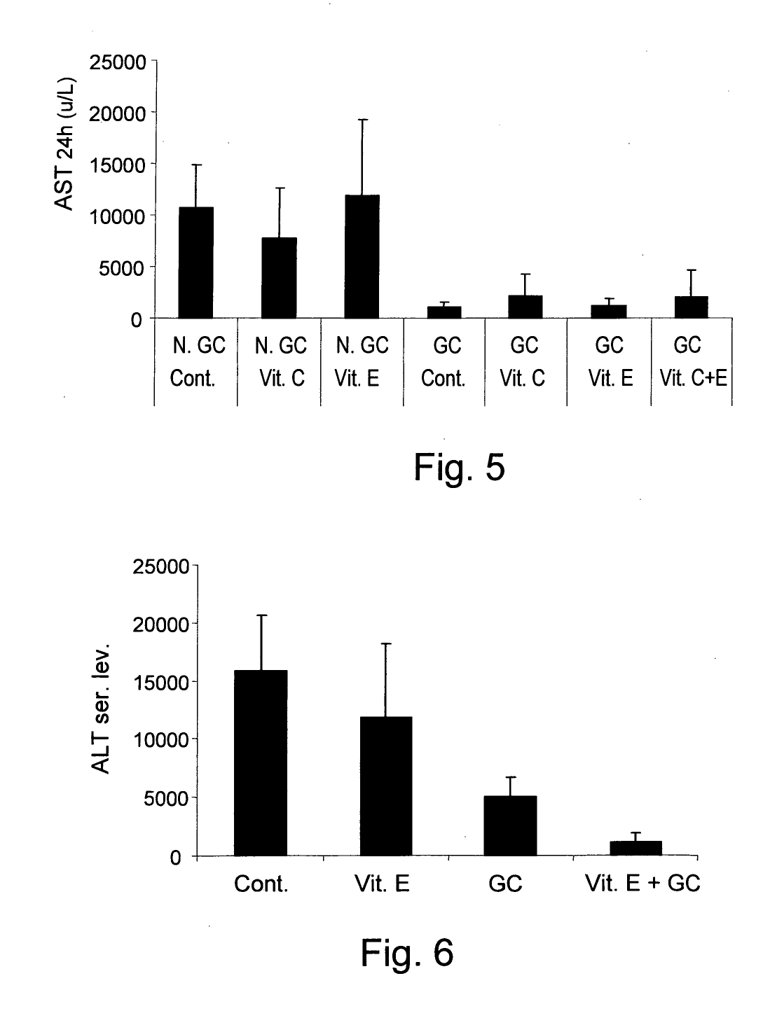

Mice of all groups (eight per group) were fasted for eight hours before oral administration of acetaminophen and for additional four hours after administration. Mice in all groups were administered orally (PO) with 10 mg/330 μl acetaminophen (in C:E gavage administration). All GC receiving groups were administered with 100 μg β-glucosylceramide (β-GC, in 30 μl of C:E) two hours before administration of acetaminophen. Mice received vitamin C, vitamin E (100 μl in gavage administration) and a combination of both (C+E), with or without the addition of GC. Animals were sacrificed after 24 hours, and liver damage was assessed in all groups by examining serum levels of ALT, AST, GSH and TNF-α. FIG. 4 clearly shows significant reduction of ALT levels when vitamins, particularly, vitamin E were combined with GC. Administration of vitamins C, E or C+E in the absence of GC had a negligible effect. Similar effect was also demonstrated when AST levels were examined, as shown by FIG. 5. Further examination of the combined treatment of GC and vitamin E, showed a clear synergistic effect as demonstrated by FIG. 6.

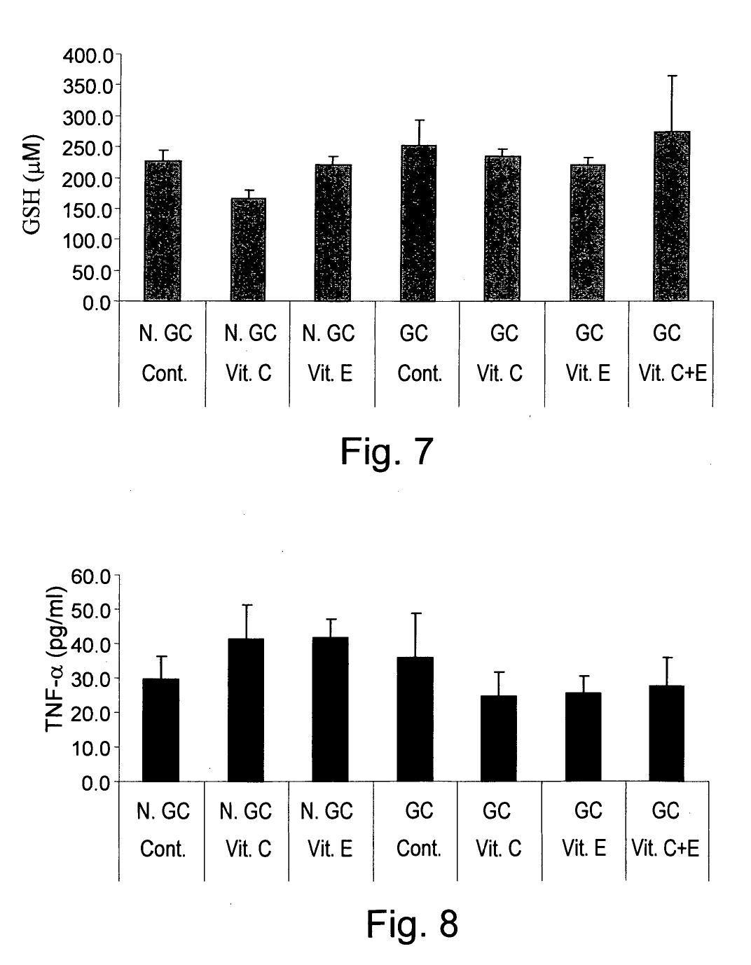

Examination of serum GSH presented by FIG. 7, showed elevation in all groups treated with a combination of GC and vitamins as compared to treatment with no GC. A trend towards a better effect of the combination was noted.

Combination of GC with the examined vitamins also showed clear effect on reducing the serum levels of TNF-α, as demonstrated by FIG. 8.

Thus, the combination clearly led to a more prominent decrease in TNF levels.

In summary, the present application clearly discloses a synergistic combination of GC and vitamin E. These results demonstrate the feasibility of using a combination of GC with vitamin E for co-administration with any type of hepatotoxic drug in order to successfully prevent or ameliorate hepatotoxic drug-induced liver injury, specifically, DILI.

1. A composition comprising a combination of at least one natural or synthetic beta-glycolipid and at least one tocopherol (vitamin E), tocotrienol or any derivatives thereof, said composition optionally further comprises at least one additional therapeutic agent and at least one pharmaceutically acceptable carrier, diluent, excipient and/or additive.

2. The composition according to claim 1, wherein said beta-glycolipid is selected from the group consisting of a glucosylceramide, a monosaccharide ceramide, a galatosylceremide, a lactosyl-ceramide, a gal-gal-glucosyl-ceramide, GM2 ganglioside, GM3 ganglioside, globoside or any synthetic or natural β-glycolipid or any derivative or combination thereof, and wherein said tocopherol (vitamin E), tocotrienol or any derivatives thereof is selected from the group consisting of: alpha-tocopherol, beta-tocopherol, gamma-tocopherol, delta-tocopherol, alpha-tocotrienol, beta-tocotrienol, gamma-tocotrienol, delta-tocotrienol and any combination thereof.

3. (canceled)

4. The composition according to claim 2, wherein said beta-glycolipid is glucosylceramide (GC) and said tocopherol (vitamin E), tocotrienol or any derivatives thereof is alpha-tocopherol.

5. The composition according to claim 1, wherein said combination further comprises at least one additional therapeutic agent selected from analgesic or antipyretic drug.

6. A pharmaceutical composition according to claim 1 for treating, preventing, ameliorating, reducing or delaying the onset of acute or chronic toxic effect of an analgesic or an antipyretic drug or any type of liver insult selected from infectious metabolic, toxic, immune, or perfusion or blood flow related hepatic injury in a subject in need thereof, comprising as an active ingredient a therapeutically effective amount of a combination of at least one natural or synthetic beta-glycolipid and at least one tocopherol (vitamin E), tocotrienol or any derivatives thereof, and optionally at least one additional therapeutic agent, with a pharmaceutically acceptable carrier.

7. The pharmaceutical composition according to claim 6, wherein said analgesic drug is acetaminophen (paracetamol).

8. The pharmaceutical composition according to claim 6, wherein said acute or chronic toxic effect is any one of drug induced liver injury (DILI), drug-induced acute steatosis, cytotoxic hepatocellular injury, acute liver failure (ALF), reperfusion injury, ischemic liver disease and acute cholestatic injury.

9. The pharmaceutical composition according to claim 6, wherein said beta-glycolipid is glucosylceramide (GC) and said tocopherol (vitamin E), tocotrienol or any derivatives thereof is alpha-tocopherol.

10. The pharmaceutical composition according to claim 6, wherein said combination further comprises at least one additional therapeutic agent selected from analgesic or antipyretic drug.

11. A method of treating, preventing, ameliorating, reducing or delaying the onset of acute or chronic toxic effect of an analgesic or an antipyretic drug or any type of liver insult selected from infectious metabolic, toxic, immune, or perfusion or blood flow related hepatic injury, in a subject in need thereof comprising the step of administering a therapeutically effective amount of a combination of at least one natural or synthetic beta-glycolipid and at least one tocopherol (vitamin E), tocotrienol or any derivatives thereof, before, simultaneously with, after or any combination thereof, administration of said drug to said subject.

12. The method according to claim 11, wherein said analgesic drug is acetaminophen (paracetamol).

13. The method according to claim 12, wherein said acute or chronic toxic effect is any one of drug induced liver injury (DILI), drug-induced acute steatosis, cytotoxic hepatocellular injury, acute liver failure (ALF), reperfusion injury, ischemic liver disease and acute cholestatic injury.

14. The method according to claim 13, wherein said beta-glycolipid is glucosylceramide (GC) and said tocopherol (vitamin E), tocotrienol or any derivatives thereof is alpha-tocopherol.

15. The method according to claim 12, wherein said GC-alpha-tocopherol combination is administered before the administration of acetaminophen to said subject, simultaneously with the administration of acetaminophen to said subject or after the administration of acetaminophen to said subject.

16. (canceled)

17. The method according to claim 15, wherein said simultaneous administration is performed by administering a combined composition comprising GC, alpha-tocopherol and acetaminophen.

18. (canceled)

19. (canceled)

20. (canceled)

21. (canceled)

22. (canceled)

23. A pharmaceutical unit dosage form comprising at least one natural or synthetic beta-glycolipid, at least one tocopherol (vitamin E), tocotrienol or any derivatives thereof and optionally, at least one additional therapeutic agent and a pharmaceutically acceptable carrier or diluent.

24. A kit for achieving a therapeutic effect in a subject suffering from acute or chronic toxic effect of an analgesic or an antipyretic drug or any type of liver insult selected from infectious metabolic, toxic, immune, or perfusion or blood flow related hepatic injury, said kit comprises:
(a) at least one natural or synthetic beta-glycolipid or a pharmaceutically acceptable derivative thereof and a pharmaceutically acceptable carrier or diluent, optionally in a first unit dosage form;
(b) at least one tocopherol (vitamin E), tocotrienol or any derivatives thereof, and a pharmaceutically acceptable carrier or diluent, optionally, in a second unit dosage form; and
(c) container means for containing said first and second dosage forms.

25. The kit according to claim 24, wherein said acute or chronic toxic effect is any one of drug induced liver injury (DILI), drug-induced acute steatosis, cytotoxic hepatocellular injury, acute liver failure (ALF), reperfusion injury, ischemic liver disease and acute cholestatic injury.

26. The kit according to claim 24, wherein said beta-glycolipid is glucosylceramide (GC) and said tocopherol (vitamin E), tocotrienol or any derivatives thereof is alpha-tocopherol.

27. (canceled)

28. A method of treating, preventing, ameliorating, reducing or delaying the onset of acute or chronic toxic effect of analgesic or antipyretic drug in a subject in need thereof comprising the step of administering to said subject a therapeutically effective amount of a first and a second unit dosage forms comprised in a kit according to claim 32, before, simultaneously with, after or any combination thereof, administration of said drug to said subject,

29. A method for increasing the maximum amount of acetaminophen that can be administered to a subject without exhibiting acetaminophen toxicity, comprising administering an acetaminophen toxicity inhibiting amount of a combination of at least one natural or synthetic beta-glycolipid and at least one tocopherol (vitamin E), tocotrienol or any derivatives thereof, or any composition or any kit thereof, before, simultaneously with, after or any combination thereof, administration of said acetaminophen to said subject.

30. A method of inducing at least one T regulatory cell in a subject in need thereof comprising the step of administering to said subject a therapeutically effective amount of at least one of:
(a) a combination of at least one natural or synthetic beta-glycolipid and at least one tocopherol (vitamin E), tocotrienol or any derivatives thereof;
(b) an immune-cell treated with any one of (a) or with any composition comprising the same;
(c) an immune-cell obtained from a subject treated with any one of (a), (b) or any combination or mixtures thereof or any composition comprising the same; and
(d) a composition comprising any one of (a), (b), (c) or any combinations or mixtures thereof, said composition optionally further comprises at least one pharmaceutically acceptable carrier, diluent, excipient and/or additive.
